SEQUENCE LISTING (41 sequences)

SEQ ID NO: 1 - RNA, 21 nt, Artificial Sequence
  Description of Artificial Sequence Synthetic oligonucleotide
agagguaguagguugcauagu

SEQ ID NO: 2 - RNA, 22 nt, Artificial Sequence
  Description of Artificial Sequence Synthetic oligonucleotide
uagcagcacauaaugguuugug

SEQ ID NO: 3 - RNA, 22 nt, Artificial Sequence
  Description of Artificial Sequence Synthetic oligonucleotide
uagcagcacguaaauauuggcg

SEQ ID NO: 4 - RNA, 22 nt, Artificial Sequence
  Description of Artificial Sequence Synthetic oligonucleotide
ucccugagacccuaacuuguga

SEQ ID NO: 5 - DNA, 22 bp, Artificial Sequence
  Description of Artificial Sequence Synthetic oligonucleotide
tgaggtagtaggttgtatagtt

SEQ ID NO: 6 - DNA, 22 bp, Artificial Sequence
  Description of Artificial Sequence Synthetic oligonucleotide
tgaggtagtagattgtatagtt

SEQ ID NO: 7 - DNA, 19 bp, Artificial Sequence
  Description of Artificial Sequence Synthetic oligonucleotide
tgaggtagtagtttgtgct

SEQ ID NO: 8 - DNA, 22 bp, Artificial Sequence
  Description of Artificial Sequence Synthetic oligonucleotide
tgaggtagtagtgtgtacagtt

SEQ ID NO: 9 - DNA, 22 bp, Artificial Sequence
  Description of Artificial Sequence Synthetic oligonucleotide
tgaggtagtagtttgtacagta

SEQ ID NO: 10 - DNA, 21 bp, Artificial Sequence
  Description of Artificial Sequence Synthetic oligonucleotide
agaggtagtaggttgcatagt

SEQ ID NO: 11 - DNA, 21 bp, Artificial Sequence
  Description of Artificial Sequence Synthetic oligonucleotide
tgaggtaggaggttgtatagt

SEQ ID NO: 12 - DNA, 22 bp, Artificial Sequence
  Description of Artificial Sequence Synthetic oligonucleotide
tgaggtagtaggttgtatggtt

SEQ ID NO: 13 - DNA, 22 bp, Artificial Sequence
  Description of Artificial Sequence Synthetic oligonucleotide
tgaggtagtaggttgtgtggtt

SEQ ID NO: 14 - DNA, 22 bp, Artificial Sequence
  Description of Artificial Sequence Synthetic oligonucleotide
tgaggtagtaagttgtattgtt

SEQ ID NO: 15 - DNA, 22 bp, Artificial Sequence
  Description of Artificial Sequence Synthetic oligonucleotide
tgaggtagtatgtaatattgta

SEQ ID NO: 16 - DNA, 18 bp, Artificial Sequence
  Description of Artificial Sequence Synthetic oligonucleotide
taacgcacagatacgact

SEQ ID NO: 17 - DNA, 19 bp, Artificial Sequence
  Description of Artificial Sequence Synthetic oligonucleotide
catagcttgatcgattatc

SEQ ID NO: 18 - DNA, 77 bp, Artificial Sequence
  Description of Artificial Sequence Synthetic oligonucleotide
taacgcacagatacgactagagttcacactatgcaacctactacctcttgctacctgagataatcgatcaagctatg

SEQ ID NO: 19 - RNA, 21 nt, Artificial Sequence
  Description of Artificial Sequence Synthetic oligonucleotide
agagguaguagguugcauagu

SEQ ID NO: 20 - DNA, 39 bp, Artificial Sequence
  Description of Artificial Sequence Synthetic oligonucleotide
taacgcacagatacgactagagttcacactatgcaacct

SEQ ID NO: 21 - DNA, 49 bp, Artificial Sequence
  Description of Artificial Sequence Synthetic oligonucleotide
taacgcacagatacgactagagtgttgaatagatcacactatgcaacct

SEQ ID NO: 22 - DNA, 49 bp, Artificial Sequence
  Description of Artificial Sequence Synthetic oligonucleotide
taacgcacagatacgactagagtgttgaatagttcacactatgcaacct

SEQ ID NO: 23 - DNA, 92 bp, Artificial Sequence
  Description of Artificial Sequence Synthetic oligonucleotide
taacgcacagatacgactagagttcacactatgcaacctactacctcttcagagcattctactaagtcactgagataatcgatcaagctatg

SEQ ID NO: 24 - DNA, 53 bp, Artificial Sequence
  Description of Artificial Sequence Synthetic oligonucleotide
actacctcttcagagcattctactaagtcactgagataatcgatcaagctatg

SEQ ID NO: 25 - DNA, 21 bp, Artificial Sequence
  Description of Artificial Sequence Synthetic oligonucleotide
gtgacttagtagaatgctctg

SEQ ID NO: 26 - DNA, 78 bp, Artificial Sequence
  Description of Artificial Sequence Synthetic oligonucleotide
taacgcacagatacgactagagttccacacaaaccattatgtgctgctaaactacctgagataatcgatcaagctatg

SEQ ID NO: 27 - RNA, 22 nt, Artificial Sequence
  Description of Artificial Sequence Synthetic oligonucleotide
uagcagcacauaaugguuugug

SEQ ID NO: 28 - DNA, 38 bp, Artificial Sequence
  Description of Artificial Sequence Synthetic oligonucleotide
taacgcacagatacgactagagttccacacaaaccatt

SEQ ID NO: 29 - DNA, 40 bp, Artificial Sequence
  Description of Artificial Sequence Synthetic oligonucleotide
atgtgctgctaaactacctgagataatcgatcaagctatg

SEQ ID NO: 30 - DNA, 78 bp, Artificial Sequence
  Description of Artificial Sequence Synthetic oligonucleotide
taacgcacagatacgactagagttccacgccaatatttacgtgctgctaaactacctgagataatcgatcaagctatg

SEQ ID NO: 31 - RNA, 22 nt, Artificial Sequence
  Description of Artificial Sequence Synthetic oligonucleotide
uagcagcacguaaauauuggcg

SEQ ID NO: 32 - DNA, 39 bp, Artificial Sequence
  Description of Artificial Sequence Synthetic oligonucleotide
taacgcacagatacgactagagttccacgccaatattta

SEQ ID NO: 33 - DNA, 39 bp, Artificial Sequence
  Description of Artificial Sequence Synthetic oligonucleotide
cgtgctgctaaactacctgagataatcgatcaagctatg

SEQ ID NO: 34 - DNA, 77 bp, Artificial Sequence
  Description of Artificial Sequence Synthetic oligonucleotide
taacgcacagatacgactagtattcctcacaagttagggtctcagggaaactacatcagataatcgatcaagctatg

SEQ ID NO: 35 - RNA, 22 nt, Artificial Sequence
  Description of Artificial Sequence Synthetic oligonucleotide
ucccugagacccuaacuuguga

SEQ ID NO: 36 - DNA, 38 bp, Artificial Sequence
  Description of Artificial Sequence Synthetic oligonucleotide
taacgcacagatacgactagtattcctcacaagttagg

SEQ ID NO: 37 - DNA, 39 bp, Artificial Sequence
  Description of Artificial Sequence Synthetic oligonucleotide
gtctcagggaaactacatcagataatcgatcaagctatg

SEQ ID NO: 38 - RNA, 22 nt, Artificial Sequence
  Description of Artificial Sequence Synthetic oligonucleotide
ugagguaguagguuguauaguu

SEQ ID NO: 39 - RNA, 22 nt, Artificial Sequence
  Description of Artificial Sequence Synthetic oligonucleotide
ugagguaguagguugugugguu

SEQ ID NO: 40 - RNA, 22 nt, Artificial Sequence
  Description of Artificial Sequence Synthetic oligonucleotide
ugagguaguagguuguaugguu

SEQ ID NO: 41 - RNA, 21 nt, Artificial Sequence
  Description of Artificial Sequence Synthetic oligonucleotide
ugagguaggagguuguauagu

(19) United States
(12) Patent Application Publication
(10) Pub. No.: US 2006/0211000 A1
(43) Pub. Date: Sep. 21, 2006

(54) Methods, compositions, and kits for detection of microRNA

(76) Inventors: Joseph A. Sorge, Del Mar, CA (US); Rebecca L. Mullinax, San Diego, CA (US)

Correspondence Address: LATIMER IP LAW, LLP, 13873 PARK CENTER ROAD, SUITE 122, HERNDON, VA, 20171, US

(21) Appl. No.: 11/084,082

(22) Filed: Mar. 21, 2005

(51) Int. Cl. C12Q 1/68; C12P 19/34

(52) U.S. Cl. 435/6; 435/91.2

(57) ABSTRACT

The present invention provides methods, nucleic acids, compositions, and kits for detecting microRNA (miRNA) in samples. The methods comprise ligating two oligonucleotides together in an miRNA mediated fashion, and detection of the ligation product. The methods can further comprise amplification of the ligation product, such as by PCR. The nucleic acids, compositions, and kits typically comprise some or all of the components necessary to practice the method of the invention.

### BACKGROUND OF THE INVENTION

1. Field of the Invention

The present invention relates to the field of molecular biology. More particularly, the present invention relates to detection of microRNA (miRNA) molecules using nucleic acid ligation.

2. Description of Related Art

MicroRNA (miRNA) are small RNA molecules that are expressed as pol II transcripts in eukaryotic organisms from fission yeasts to higher organisms. They have been shown to regulate gene expression, mRNA splicing, and histone formation. They also have been shown to have tissue-specific and developmental-specific expression patterns. Thus, these small RNA molecules are of great interest in elucidation of biological processes, disease states, and development.

miRNA are expressed as pol II transcripts as relatively long RNA molecules called pri-miRNA. These pri-miRNA have a 5′ cap and a poly-A tail, like other RNA transcripts. The pri-miRNA are subsequently processed into hairpin-loop structures in the nucleus, then the hairpin structure is cleaved at the base of the stem by Drosha to form double-stranded molecules referred to as pre-miRNA. The pre-miRNA are exported to the cytoplasm by exportin 5, where they are processed by cleavage by Dicer into short (17-25 nucleotide) double-stranded RNA molecules. The strand of the pre-miRNA with less 5′ stability then can become bound to the RNA interference silencing complex (RISC) and effect mRNA regulation by binding at the 3′ untranslated region (3′ UTR) of certain mRNA. Binding results in either cleavage of the target mRNA if there is 100% complementarity between the miRNA and the target RNA (RNAi) or down-regulation of expression (without cleavage) by binding to the target mRNA and blocking translation if there is less than 100% complementarity between the miRNA and the target RNA. A useful resource for miRNA information is available from the Sanger Institute, which maintains a registry of miRNA.

miRNA have been found in both coding and non-coding sequences within the genome. The have also been found to exist oriented in both the sense or anti-sense direction with regard to the particular gene in which they are located. Furthermore, various miRNA have been detected as single copies in a gene or mRNA transcript, or as multiple copies in a gene or mRNA transcript. Additionally, more than one miRNA has been detected in an mRNA transcript.

Expression of miRNA in various cells has been estimated at less than 1,000 copies to more than 500,000 copies. In mammalian cells, miRNA primarily interact with the 3′ UTR of genes to inhibit translation of the encoded mRNA. Studies have shown that differential miRNA expression occurs in cancerous and non-cancerous tissues. Thus, detection of miRNA expression might be useful in diagnostics, including diagnosis of cancerous conditions.

Various techniques have been developed to detect new miRNA and to attempt to quantitate known miRNA in samples or tissues. Many of the studies performed to date have focused on determining the relative levels of miRNA expression. In a common technique, inserts from miRNA are ligated into a vector and then sequenced. In other techniques, Northern blotting is used to identify expression of miRNA. In general, Northern blotting techniques for studies of miRNA include lysing a cell sample, enriching for low molecular weight RNA, generating a typical Northern blot, hybridizing to a labeled probe, which is complementary to an miRNA of interest, and determining the relative molecular weights of detected species to gain a general understanding of the relative amounts of pri-miRNA, pre-miRNA, and miRNA in the original sample.

Studies using Northern blotting typically focus on detection and confirmation of expression of predicted miRNA, and often attempt to quantitate miRNA expression in samples, particularly to determine tissue and time point specific miRNA expression. Studies using Northern blotting have also been performed in attempts to determine ratios of pri-miRNA, pre-miRNA, and miRNA in samples. Although studies have been performed to elucidate expression of miRNA, currently little is known about the regulation of processing of miRNA. Expression studies indicate that there is differential expression of some miRNA in disease states as compared to normal states, there is currently no information available about processing, and the possibility of differential processing, of miRNA in diseased tissues. To date, studies have indicated that processing of miRNA is regulated in some way, but the precise mechanisms have not been elucidated. It is believed that very little, if any, pri-miRNA is long-lived (based on levels of detection) in normal cells.

In addition to Northern blot techniques for analysis of miRNA, in silico predictions are widely used to study miRNA expression. Computer algorithms have been developed and implemented to identify new miRNA. These in silico methods generally include scanning an organism's genome for sequences that have the potential to form hairpins. Sequences that are identified are then scanned for complementarity to 3′ UTR and compared to known homologs. Potential targets are then confirmed by bench experiments, such as through Northen blot experiments.

Microarrays have also been used to identify miRNA. Microarrays have been found to be best suited for identification of expressed miRNA sequences, and to measure the relative expression levels of miRNA. In general, microarray methods include spotting oligonucleotides that are complementary to known miRNA sequences on an array, generating fluorescence-labeled miRNA, and exposing the labeled miRNA to the array to determine if any miRNA of interest are present. Microarrays have been used to validate predicted miRNA, to discover homologs of known miRNA, to identify and monitor expression of a given miRNA in a tissue and/or over a time course, and to study miRNA processing.

A number of techniques have been developed over the last 30 years to detect nucleic acids of interest. Such techniques include everything from basic hybridization of a labeled probe to a target sequence (e.g., Southern blotting) to quantitative polymerase chain reaction (QPCR) to detect two or more target sequences with multiple amplification primers and/or detection probes. Amplification is now commonly used in techniques designed to identify small quantities of a target nucleic acid in a sample. Although PCR is the most common method of amplifying nucleic acid targets in samples, other techniques, such as the ligase chain reaction (LCR) and strand displacement amplification (SDA) are also commonly used.

DNA ligases have long been used to distinguish single nucleotide variations in DNA sequences by ligation of DNA oligonucleotides annealed to the DNA sequence of interest under conditions where the presence of a terminal mismatch in the DNA oligonucleotides causes less efficient ligation than is seen when perfectly matched DNA oligonucleotides are used. These methods are directed toward detecting single nucleotide polymorphisms (SNPs) in a double-stranded genomic DNA template at the ligation point. One such method, described in U.S. Pat. Nos. 6,027,889, 6,268,148, and 6,797,470, is directed toward the detection of SNPs in genomic DNA. In one preferred embodiment, these patents describe the use of a primer having a detectable reporter label. However, these patents do not approach detection of sequences in RNA molecules.

It has also long been known that T4 DNA ligase can direct ligation of DNA oligonucleotides when annealed to an RNA molecule. For example, Hsuih et al. (Hsuih, T., et al., “Novel Ligation-Dependent PCR Assay for Detection of Hepatitis C Virus in Serum, J. Clin. Micro. 34(3):501-507, 1996) disclose the use of T4 DNA ligase to ligate two DNA oligonucleotides that are brought together as a consequence of binding to an RNA of interest (HCV RNA). Hsuih's method involves capture of the RNA followed by ligation of two probes and amplification of the ligation product. However, Hsuih does not contemplate detection of small RNA molecules, such as miRNA, and indeed cannot contemplate detection of miRNA in view of the publication of the method five years before the discovery of miRNA.

Although methods of using T4 DNA ligase to detect nucleic acids has been known for some time, the methods have proved to be inefficient when detecting RNA, and therefore are not widely practiced. To address these limitations, U.S. Published Patent Application 2004/0106112 describes an optimal reaction medium useful in ligating DNA oligonucleotides when annealed to an RNA template. The optimal reaction conditions are used to distinguish RNA sequence variants. While the conditions disclosed in that patent application are effective in directing ligation, the application does not recognize that other conditions may be suitable for detection of miRNA. Indeed, the published patent application, which has a filing date prior to the discovery of miRNA, does not even contemplate detection of miRNA.

While numerous techniques and reagents are available for detection and analysis of miRNAs, there still exists a need in the art for methods of miRNA detection that also quantitate the miRNA in the sample, methods that are less labor-intensive than those currently available, and methods that can be used to validate the various current techniques, such as microarray results.

### SUMMARY OF THE INVENTION

The present invention provides a system for detecting nucleic acids in a sample. The system has multiple aspects, including methods, nucleic acids, compositions, and kits. In general, the nucleic acids, compositions, and kits comprise materials that are useful in carrying out the methods of the invention or are produced by the methods, and that can be used to detect nucleic acids of interest that are present in samples.

In a first aspect, the invention provides a method of detecting microRNA (miRNA) molecules, including its precursor miRNAs (pri-miRNA and pre-miRNA), that are present in a sample. As used herein, miRNA are those molecules that meet the criteria of the Sanger Institute miRNA Registry (and precursors to those molecules). Thus, this aspect of the invention provides methods for determining the presence or absence of miRNA molecules in a sample. The method generally comprises providing two ligator oligonucleotides, providing a sample containing or suspected of containing an miRNA, combining the ligator oligonucleotides and sample to make a mixture, exposing the mixture to conditions that permit ligation of the two oligonucleotides to form a single oligonucleotide product, also referred to herein as a ligation product, and detecting the presence or absence of the ligation product. In general, the presence of an miRNA to which the ligator oligonucleotides bind causes the ligator oligonucleotides to be brought into close enough proximity for their ligation to each other, resulting in a nucleic acid product that can be detected more easily than the miRNA of interest, and which, in embodiments, can be in greater abundance than the miRNA of interest. In certain embodiments, the ligation product is amplified to further increase the amount of ligation product and enhance detection.

In a second aspect, nucleic acids are provided. The nucleic acids are generally nucleic acids that are useful in performing at least one embodiment of the method of the invention, or are created by at least one embodiment of the invention. The nucleic acids thus may be ligator oligonucleotides, ligation products (also called oligonucleotide products), amplification primers, miRNA (for use as positive controls), and other nucleic acids that can serve as controls for one or more steps of the method.

In a third aspect, compositions are provided. Typically, the compositions comprise one or more component that is useful for practicing at least one embodiment of the method of the invention, or is produced through practice of at least one embodiment of the method of the invention. The compositions thus may comprise two or more ligator oligonucleotides according to the invention. They may also comprise a ligation product of two ligator oligonucleotides. They also may comprise two or more amplification primers, at least one ligase, at least one polymerase, and/or one or more detectable labels.

In a fourth aspect, kits are provided. Kits according to the invention provide at least one component that is useful for practicing at least one embodiment of the method of the invention. Thus, a kit according to the invention can provide some or all of the components necessary to practice at least one embodiment of the method of the invention. In typical embodiments, a kit comprises at least one container that contains a nucleic acid of the invention. In various specific embodiments, the kit comprises all of the nucleic acids needed to perform at least one embodiment of the method of the invention.

### BRIEF DESCRIPTION OF THE DRAWINGS

The accompanying drawings, which are incorporated in and constitute a part of this specification, illustrate several embodiments of the invention, and together with the written description, serve to explain certain principles or details of various embodiments of the invention.

FIG. 1 depicts a general scheme for one embodiment of a method according to the present invention.

FIG. 2 depicts a general scheme for embodiments of the method in which amplification of the ligation product is performed using QPCR.

FIG. 3 depicts a general scheme for a ligation-QPCR assay according to embodiments of the invention.

FIG. 4 depicts one embodiment of an up ligator oligonucleotide of the invention, which is specific for the let-7d miRNA.

FIG. 5 depicts the up ligator oligonucleotide of FIG. 4, showing the regions of self-complementarity.

FIG. 6 depicts one embodiment of a down ligator oligonucleotide of the invention, which is specific for the let-7d miRNA.

FIG. 7 depicts the down ligator of FIG. 6, showing the region of self-complementarity.

FIG. 8 depicts one embodiment of an up ligator oligonucleotide of the invention, which is specific for the let-7d miRNA and has 8 bases of self-complementarity.

FIG. 9 depicts the up ligator of FIG. 8, showing the region of self-complementarity.

FIG. 10 depicts one embodiment of an up ligator oligonucleotide of the invention, which is specific for the let-7d miRNA and has 9 bases of self-complementarity.

FIG. 11 depicts the up ligator of FIG. 10, showing the region of self-complementarity.

FIG. 12 depicts an up ligator according to one embodiment of the invention, which is specific for the miR-16 miRNA.

FIG. 13 depicts the up ligator of FIG. 12, showing the region of self-complementarity.

FIG. 14 depicts a down ligator according to one embodiment of the invention, which is specific for the miR-16 miRNA.

FIG. 15 depicts the down ligator of FIG. 14, showing the region of self-complementarity.

FIG. 16 depicts an up ligator according to one embodiment of the invention, which is specific for the miR-15a miRNA.

FIG. 17 depicts the up ligator of FIG. 16, showing the regions of self-complementarity.

FIG. 18 depicts a down ligator according to one embodiment of the invention, which is specific for the miR-15a miRNA.

FIG. 19 depicts the down ligator of FIG. 18, showing the region of self-complementarity.

FIGS. 20A-C depict design and sequential steps in creation of ligator oligonucleotides according to an embodiment of the invention.

FIG. 21 depicts a standard curve for QPCR amplification of the let-7D ligation product (provided as an oligonucleotide product).

FIG. 22 depicts a standard curve generated with let-7D miRNA as a template.

FIG. 23 depicts a ligation-QPCR assay of one embodiment of the invention to detect let-7d and miR-16 in a sample that had been enriched for miRNA from HeLa S3 tissue culture cells.

FIG. 24 depicts detection of let-7D, miR-15a, and miR-16 in various cell lines and UHRR.

FIG. 25 depicts gel analysis of ligation products using hydrolysis probe and hairpin ligators.

FIG. 26 depicts the effect of Perfect Match PCR Enhancer on QPCR according to an embodiment of the invention.

FIG. 27 depicts a general scheme for embodiments of the method in which a hydrolysis probe is used for multiplexing.

FIG. 28 depicts a general scheme for embodiments of the method in which a hairpin probe is used.

FIG. 29 depicts a general scheme for embodiments of the method in which blocking oligonucleotides are used.

### DETAILED DESCRIPTION OF VARIOUS EMBODIMENTS OF THE INVENTION

Reference will now be made in detail to various exemplary embodiments of the invention, examples of which are illustrated in the accompanying drawings.

In recent years, the study of the non-coding class of RNA termed microRNA (miRNA) has grown significantly because of their role in post-transcriptional gene regulation. The identification of novel miRNA sequences has often involved computational approaches, with validation by Northern blot analysis or microarray analysis. Traditional QRT-PCR approaches cannot be implemented for mature miRNA detection because the approximately 22 nucleotide sequences are not of sufficient length for primer extension by the reverse transcriptase. Herein we describe a ligation method, which in embodiments is a ligation-QPCR method, for the detection of miRNA sequences. The method utilizes a miRNA-dependent ligation step, which can result in a detectable product or the formation of a template for amplification, such as by QPCR. The inherent sequence specificity in ligations has allowed for the estimation and quantitation of miRNA expression levels in various cell lysate samples. Potential applications of this technique include use as a tool for miRNA discovery or as a method for validation of microarray or Northern blot data.

miRNAs are a class of non-coding RNA sequences that range in length from 17 to 24 nucleotides (nt). There are currently 222 Homo sapiens miRNA sequences registered in the Sanger Institute's miRNA Registry. Mature miRNA sequences result from a two-step processing of pri-miRNA transcripts by Drosha to produce the pre-miRNA intermediate, followed by Dicer to form the mature miRNA. In the mature form, the miRNA binds to the 3′-untranslated region (UTR) of mRNA targets to form an RNAi-induced silencing complex (RISC), which can inhibit translation by a number of methods. miRNAs have been linked to several diverse functions, including developmental timing, as well as a number of diseases including cancer.

Several miRNA are only expressed in specific developmental stages or in specific cells. Exemplary embodiments of the present invention relate to a subset of miRNA sequences, whose expression levels are found to vary between normal and cancerous cells, and the development of a system for monitoring their expression. The development of a system for monitoring miRNA expression levels can allow for a better understanding of their biological roles and thereby their potential role in cancer or other diseases or disorders. The correlation between miRNA expression data and its link to disease state in the body may ultimately play a key role in early diagnosis.

In a first aspect, the invention provides a method of detecting microRNA (miRNA) molecules that are present in a sample. The method generally comprises providing two ligator oligonucleotides, providing a sample containing or suspected of containing an miRNA, combining the ligator oligonucleotides and sample to make a mixture, exposing the mixture to conditions that permit ligation of the two oligonucleotides to form a single ligation product, and detecting the presence or absence of ligation product.

Providing, whether it be in reference to ligator oligonucleotides, a sample, or any other substance used in the method, can be any act that results in a particular substance being present in a particular environment. Broadly speaking, it can be any action that results in the practitioner obtaining and having in possession the substance of interest in a form suitable for use in the present method (the term “assay” being used herein interchangeable on occasion). Those of skill in the art are aware of numerous actions that can achieve this result. In addition, non-limiting examples are provided throughout this disclosure. For example, providing can be adding a substance to another substance to create a composition. It can include mixing two or more substances together to create a composition or mixture. It can also include isolating a substance or composition from its natural environment or the environment from which it came. Providing likewise can include obtaining a substance or composition in a purified or partially purified form from a supplier or vendor. Additionally, providing can include obtaining a sample suspected of containing an miRNA of interest, removing a portion for use in the present method, and maintaining the remaining amount of sample in a separate container from the portion to be used in the present method.

Combining substances or compositions in the method means bringing two or more substances, compositions, components, etc. into contact such that a single composition of the two results. Any act that provides such a result is encompassed by this term, and those of skill in the art are aware of numerous ways to achieve the result. A non-limiting example of actions that are considered combining is adding a composition comprising one or more ligator oligonucleotides to an aqueous sample containing or suspected of containing an miRNA species. Combining can also include actions that result in the combination being a homogeneous or otherwise mixed composition in which substances of one starting material are interspersed with substances from one or more other starting materials. Thus, combining can include mixing to make a mixture. It can therefore include stirring, repetitive pipetting of the combination, inverting a container containing the combination, shaking the combination, vortexing the combination, or even permitting the combination to stand for a sufficient amount of time for random diffusion to effect partial or complete mixing. Mixing can also include any action that might be required to maintain a homogeneous or nearly homogeneous composition, including, but not limited to performing a new action or repeating one or more actions that resulted in an initial mixture.

The method comprises exposing a mixture comprising two ligator oligonucleotides and a sample containing or suspected of containing an miRNA to conditions that permit ligation of the two oligonucleotides to form a single ligation product. Any suitable amount of ligator oligonucleotides may be used. Exemplary concentrations include 0.01 uM, 0.1 uM, and 0.4 uM. Each ligator oligonucleotide may be added in a concentration that is independently selected from any other ligator oligonucleotide. According to the method of the invention, if one or more molecules of an miRNA species of interest (also referred to herein as the “target miRNA”) are present in the sample, this exposing results in ligation of two ligator oligonucleotides to form a single, relatively long oligonucleotide product. While theoretically, the method can be practiced with literally two ligator oligonucleotides, by reference to the oligonucleotides, it is envisioned that numerous identical copies of each will be provided each time the method is performed, as is typical for methods performed in the molecular biology field. Thus, reference throughout this disclosure to a certain number of nucleic acids, whether they be ligator oligonucleotides, ligation products, amplification primers, amplification products, or any other nucleic acid, is in reference to the particular identity of the nucleic acid, and encompasses one or multiple exact or essentially exact copies of that nucleic acid.

In situations where the target miRNA is not present in the sample, a lesser amount of ligation between the two ligator oligonucleotides occurs, and only background levels are detected. The amount of ligation seen in the presence of the target miRNA is significantly higher than the amount seen in the absence of it. In embodiments, no ligation is seen in the absence of the target miRNA. By no ligation, it is meant that the amount of ligation that occurs is undetectable or not significantly different than the amount that can be detected in a composition that lacks the sample, but is otherwise identical. Thus, the presence of the target miRNA in the sample significantly increases the amount of ligation of the two ligator oligonucleotides above the level that would occur in the absence of the target miRNA. Accordingly, the method of the invention is capable of detecting the presence or absence of a target miRNA.

Those of skill in the art are cognizant of numerous techniques for ligating two nucleic acids. Any suitable technique and set of conditions may be used in practicing the present method. Thus, any of the following ligases, or mutants thereof, may be used (in accordance with conditions known in the art as suitable for ligation activity of the particular ligase): E. coli DNA ligase, T4 DNA ligase, Pfu DNA ligase, Tfi DNA ligase, and DNA ligases from Chorella, Bacillus stearothermophilus, Thermus scotoductis, and Thermus aquaticus. In embodiments, two or more ligases may be included in the ligation reaction, each supplying one or more advantageous activities, such as thermostability, specificity for substrate (DNA, RNA, etc.), salt optimum, tolerance for mismatches at the ligation junction, and the like).

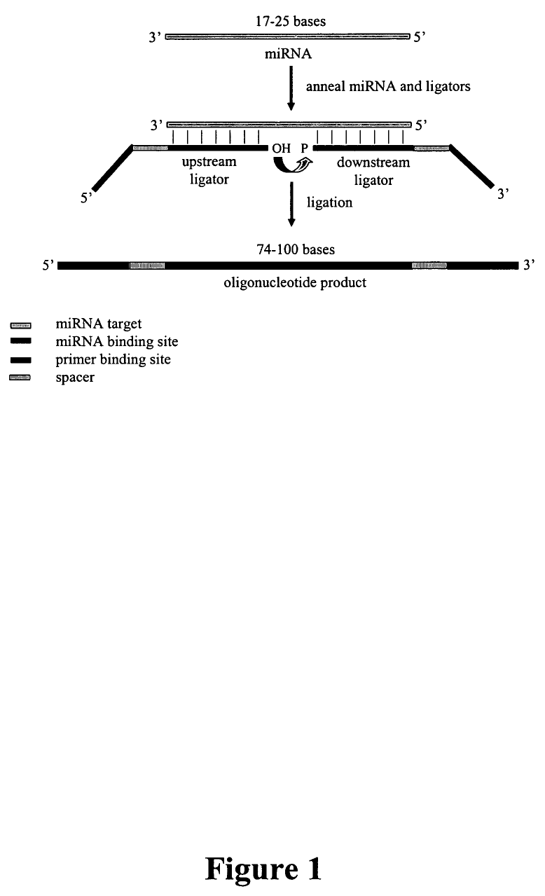

The method of the invention involves the miRNA target bringing the two ligator oligonucleotides into close enough proximity for ligation of the two oligonucleotides to occur. The general scheme is depicted in FIG. 1. As discussed in detail below, each ligator oligonucleotide comprises a sequence that is specific for a portion of the target miRNA such that, upon binding of the two ligator oligonucleotides to the target miRNA, the 5′ end of one ligator oligonucleotide is adjacent to the 3′ end of the other, permitting ligation of the two under appropriate conditions to produce a ligation product. Due to its size, the fact that it can be labeled, the fact that it can be amplified easily, and the fact that it is deoxyribonucleic acid rather than ribonucleic acid, the ligation product can be detected more easily than the original miRNA. Thus, the present method provides a rapid, convenient, and reliable method for detecting the presence of a target miRNA in a sample.

On the other hand, in the absence of target miRNA, the two ligator oligonucleotides are not brought into close proximity by the miRNA, and will not be ligated to each other to any appreciable, significant extent. The method thus provides an indication of the absence of a target miRNA in a sample of interest.

In embodiments, the ligation reaction includes additional components to increase ligation efficiency and/or ligation specificity. Such additives include, but are not limited to, Perfect Match® PCR enhancer (Stratagene), betaine, dimethyl sulfoxide (DMSO), tetramethyl ammonium chloride (TMAC), polyethylene glycol 8000 (PEG8000), and/or polyamines (see, for example, Venkiteswaran, S., V. Vijayanathan, A. Shirahata, T. Thomas. 2004. Antisense recognition of the HER-2 mRNA: effects of phosphorothioate substitution and polyamines on DNA:RNA, RNA:RNA, and DNA:DNA duplex stability. Biochemistry. 44(1):303-312). Of particular interest are those polyamines that have been developed to enhance the effectiveness of anti-sense technology, which is dependent upon the annealing of RNA and DNA (Venkiteswaran, S., above) and the use of hybrid oligomer duplexes formed with phosphorothioate DNA (Hashem, G. M., L. Pham, M. R. Vaughan, and D. M. Gray. 1998. Hybrid oligomer duplexes formed with phosphorothioate DNAs:CD spectra and melting temperatures of S-DNA:RNA hybrids are sequence dependent but consistent with similar heteronomous conformations. Biochemistry. 37(1):61-72).

Of course, the ligation reaction may be performed under different conditions, which result in an increase in ligation efficiency and/or ligation specificity. Such conditions comprise variations in annealing temperatures and times prior to and after the addition of the ligation reagent.

In view of the shortcomings of the prior art, it has been surprisingly found that miRNA can serve as a template for bringing the two ligator oligonucleotides together, even though the miRNA is relatively small (typically about 18-25 nucleotides) and may have sequences that are disadvantageous for hybridization. In addition, it has been surprisingly found that miRNA-mediated ligation of two ligator oligonucleotides is possible even though the miRNA may contain sequences that have been shown to be disadvantageous for ligation, or the ligation conditions are sub-optimal. While others have disclosed methods of detecting DNA molecules and long RNA molecules using ligation, it previously could not be predicted that small nucleic acids, on the order of 18-25 nucleotides in length, much less ribonucleic acids of this approximate size, could be detected using a ligation technique, with or without a subsequent amplification.

The method of the invention comprises detecting the presence of a ligation product. The ligation product may be one produced from pri-miRNA, pre-miRNA, or miRNA. Detection of pri-miRNA and pre-miRNA can be through binding of ligator oligonucleotides to the miRNA sequences or other sequences present in these precursor molecules. Detection can be through any technique known in the field of molecular biology for detecting nucleic acids. Thus, it can be through agarose gel electrophoresis and staining with a nucleic acid specific stain. It can be through labeling of one or more of the ligator probes with a detectable moiety, such as a fluorescent or radioactive molecule to produce a labeled ligation product. Likewise, it can be through labeling with a member of a two-component label system, such as the digoxigenin system. Other non-limiting examples include detection based on column chromatography (e.g., size exclusion chromatography), mass spectrometry, and sequencing. Yet other non-limiting techniques include amplification of signal by enzymatic techniques and use of antibodies that are specific to a label attached to one or more nucleotides of the product to be detected. In embodiments where additional, optional steps are added to the basic method, detection can include other activities. For example, in embodiments where amplification of the ligation product is performed (see below), detection can be through real-time monitoring of luminescence/fluorescence as amplification proceeds. Those of skill in the art are well aware of the various techniques for detecting nucleic acids, and the various devices, supplies, and reagents that can be used to do so. Thus, the detection techniques, devices, supplies, and reagents need not be detailed here.

Detection can result in qualitative identification, semi-quantitative identification, or quantitative identification of the target miRNA. Qualitative detection includes detection of the presence of a ligation product or amplification product, without any correlation to an amount of target miRNA in the sample that was tested. Qualitative results enable the practitioner to conclude that the target miRNA was present in the sample, but do not enable him to ascertain the amount. Semi-quantitative detection permits not only detection of a signal, but correlation of the signal to a basal level of target miRNA in the sample that was tested. For example, it may indicate a minimum threshold amount of miRNA was present in the sample. Such a result enables the practitioner to determine if a pre-defined amount of miRNA target is present in the sample, but not to determine if less than that amount is present. Likewise, it does not enable the practitioner to determine the precise concentration or amount of miRNA in the original sample. Quantitative detection permits the practitioner to determine the amount of target miRNA present in the original sample over a wide range of amounts. In general, quantitative detection compares the amount detected to a reference or standard that is either previously generated (e.g., a standard curve) or generated at the time of the assay for the target miRNA using internal controls. Numerous techniques for performing quantitative and semi-quantitative analyses are known to those of skill in the art, and need not be detailed here. For example, those of skill in the art may consult various commercial products for suitable techniques for performing PCR, QPCR, generating standard curves, and quantitating and validating amplification results.

The method may comprise one or more additional optional steps as well. For example, nucleic acids or other substances can be purified to any extent prior to or at any time during the method, including as part of one or more steps, such as the detecting step. Likewise, inhibitors that might be present in one or more compositions can be removed by purification of the nucleic acids of the invention from the inhibitors. Such purification can be performed between two or more other steps of the method. In addition, portions of one or more compositions formed during practice of the method may be removed. These can be used for any purpose, including, but not limited to, performing control reactions to ensure that one or more steps in the method are functioning properly, assaying for one or more substances in the composition to ensure that it is present, preferably in the amount expected, and determining any other reaction parameter of interest.

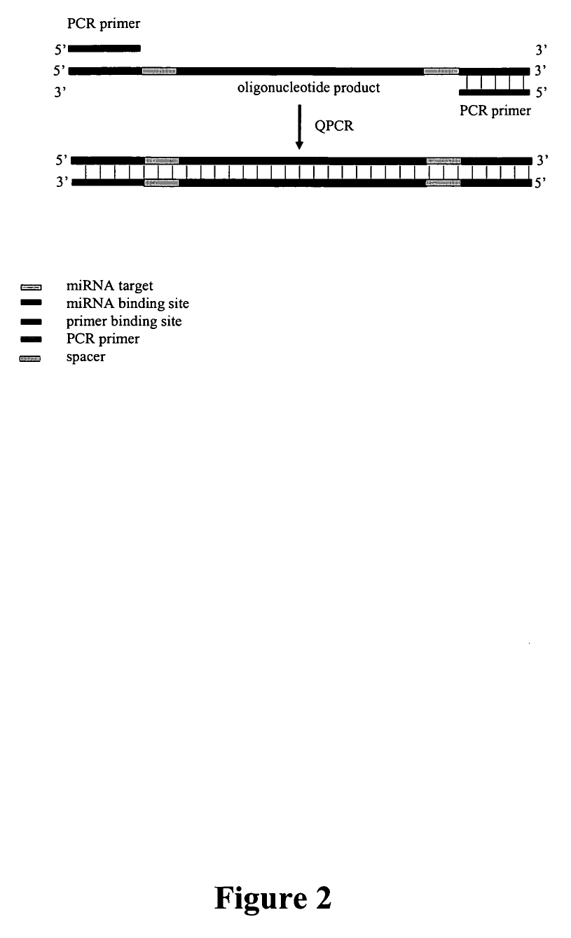

The method can comprise amplification of the ligation product prior to, or at the time of, detection. In embodiments where the ligation product is amplified, it is also referred to herein as an amplification template. Amplification of the ligation product can be performed using any suitable amplification technique, including, but not limited to, PCR and all of its variants (e.g., real-time PCR or quantitative PCR). In embodiments where amplification is included in the method, the method further comprises providing at least one amplification oligonucleotide primer, exposing the oligonucleotide ligation product, if present, to the amplification primer, and exposing the resulting mixture to conditions that permit amplification of the single oligonucleotide ligation product, if present. Of course, the ligator oligonucleotides may be used as amplification primers. However, this is not preferred. Furthermore, while it is possible to amplify the ligation product with a single amplification primer (using one of the ligator oligonucleotides as a second amplification primer), this is not preferred. A general scheme for embodiments that include amplification, including amplification with PCR, is depicted in FIGS. 2-5, for example.

The amplification primers may be exposed to the other components of the method at any time during practice of the method. Thus, they may be exposed to the other components before, at the same time as, or after exposure to the ligator oligonucleotides. Likewise, they may be exposed to the composition after one or more polymerases are exposed to the other components. Accordingly, the method of the invention can be practiced in a single tube format or a multiple tube format (i.e., all reactions can be performed in a single reaction vessel with some or all components being added together, or some reactions can be performed in one reaction vessel and others performed in a second reaction vessel). As with the ligator oligonucleotides, both amplification primers need not be exposed to the other components at the same time, although it is envisioned that they typically will be. The amplification primers may be exposed to the other components of the method after ligation of the ligator oligonucleotides has occurred (or after the conditions for ligation have been provided). Under certain circumstances, amplification primers can be added multiple times, for example prior to exposing the composition to conditions where amplification may occur, then during the amplification process. Likewise, if a sample is to be removed during practice of the method, amplification primers may be added only to the removed sample, only to the remaining composition, or both. Furthermore, multiple different primers or sets of primers may be added, either to a single composition or to different compositions resulting from removal of one or more portions from the composition. In this way, different amplification efficiencies can be determined based on different amplification primer sequences, or other information can be gathered based on other amplification parameters.

The sample can contain an miRNA (as mentioned above, included in this term are pri-miRNA and pre-miRNA) of interest or no miRNA of interest. The method of the invention is capable of determining whether an miRNA of interest or a related miRNA having identity at the site of hybridization for the ligator oligonucleotides is in the sample or not. Thus, the method can be a method of determining the presence or absence of an miRNA of interest in a sample. As discussed above, if the target miRNA is present in the sample, it will mediate ligation of the two ligator oligonucleotides, and a ligation product will be produced. This ligation product may be detected directly or subjected to amplification for enhanced detection. In the absence of the target miRNA, no significant ligation will occur, and no or an insignificant amount of ligation product will be detected.

Because the method is designed not to detect an miRNA of interest when it is not present in the sample, the practitioner may desire to perform one or more control reactions to ensure that one or more steps of the method are performed properly and/or one or more substance, component, reagent, etc. is functioning as expected. Thus, the method of the invention may optionally comprise one or more control reactions, either performed internally as part of the method in the ligation and/or amplification composition, or as one or more separate reactions performed in addition to the reactions encompassed by the general method of the invention. Thus, for example, the sample may be exposed to an miRNA of known identity (but typically a different species than the target miRNA) and to two ligator oligonucleotides that are specific for the known miRNA species. Ligation and, optionally, amplification may be performed with those control nucleic acids present to ensure that the method functioned properly, and that any lack of detectable signal from the target miRNA is due to a lack of that miRNA in the original sample, rather than due to a failure of one or more steps of the method. In a similar fashion, a known miRNA species may be detected by ligation and amplification in a separate reaction vessel that is otherwise treated identically to the reaction vessel containing the sample being tested, to monitor the functioning of the method. Other controls that are known by those of skill in the art as useful in performing ligation and/or amplification reactions may be used as well. Such controls are well known to those of skill in the art, and thus need not be detailed here. Exemplary negative controls can be used to determine the basal level (i.e., background level) of ligation (e.g., in the absence of miRNA target, the absence of any nucleic acids in a sample, the absence of ligase, the absence of polymerase, etc.) or basal level of amplification (e.g., in the absence of ligator oligonucleotides to form the ligation product, the absence of one or more amplification primers, the absence of polymerase, etc.). One may select the positive or negative controls as desired or dictated by the particular embodiment being practiced or sample being tested. Such a selection is well within the skill level of those of skill in the art.

The sample is any sample from any source that contains or is suspected of containing an miRNA of interest. It thus may be a sample from an animal, plant, or fission yeast. It can be an environmental sample, a clinical sample, a laboratory sample, or a sample from an unknown source. Likewise, a sample can be one that derives from two separate sources, which were combined to create a single sample. Combining or pooling of samples may be preferred when the method of the invention is practiced to screen a large number of samples at one time (e.g., high throughput screening). In such situations, pooling permits multiple samples to be assayed in a single reaction vessel—if a positive result is obtained, the individual samples of the pool may later be individually screened by the method to identify the one (or more) samples containing the miRNA of interest.

Additionally, methods resulting in an increase in accessibility of the miRNA for annealing are contemplated by the present invention. In the cell, miRNA might be associated with one or more of the following: one or more proteins, one or more protein complexes, mRNA, target mRNA, small nuclear (snRNA), genomic DNA, cellular membranes, and/or combinations thereof. Such methods to increase miRNA accessibility could include thermal denaturation, protein denaturation and/or removal, and membrane solubilization and/or removal.

The methods of the invention can detect miRNA having a known sequence. They likewise can detect related miRNA, which may or may not have an identical sequence to a known miRNA sequence. Thus, the methods of the invention can be methods of detecting and/or identifying two or more members of an miRNA family or detecting and/or identifying new miRNA species, or detecting and/or identifying miRNA homologs. Typically, when the method is practiced to detect new miRNA species, detection is based on use of ligator oligonucleotides that either have a sequence that is perfectly complementary to a known miRNA species or have a sequence that has high, but not perfect, complementarity to a known miRNA sequence. In either case, detection of the related miRNA can be accomplished by adjusting the ligation reaction conditions to permit hybridization of the ligator oligonucleotides to the miRNA, and permit ligation of the two ligator oligonucleotides to occur. Accordingly, the methods can detect miRNA having sequences that are 70% or greater identical to a known miRNA sequence at the region of hybridization, such as those having 80% or greater identity, 90% or greater identity 92% or greater identity, or 96% or greater identity (or any whole or fractional percentage within this range).

One advantage of the methods of the invention, be they methods of detecting a single miRNA or multiple miRNA having sequence identity, is the ability to monitor expression of certain miRNA across tissue samples or through time. It is known that certain miRNA are expressed in specific tissues or at specific times of development. In some instances, these expression patterns are correlated with disease or disorder states of the individual with which the tissue is associated. By practicing the present invention, progression or status of a disease or disorder may be monitored. Furthermore, monitoring expression of a particular miRNA or multiple miRNAs having a given level of sequence identity can permit the practitioner to identify new tissues that are affected by a certain diseases or disorders. It also can permit the practitioner to determine a new association of a disease or disorder with an miRNA or an miRNA having a certain level of sequence identity. It also can permit the practitioner to identity responses generated by tissues that are present in organisms affected by a disease or disorder. For example, monitoring of apparently healthy tissues along with diseased tissues in a person suffering from a cancer may permit the practitioner to identify cellular responses in both the diseased tissue and the healthy tissue that can be helpful in developing a treatment, or in understanding the response an organism mounts when confronted with a disease state.

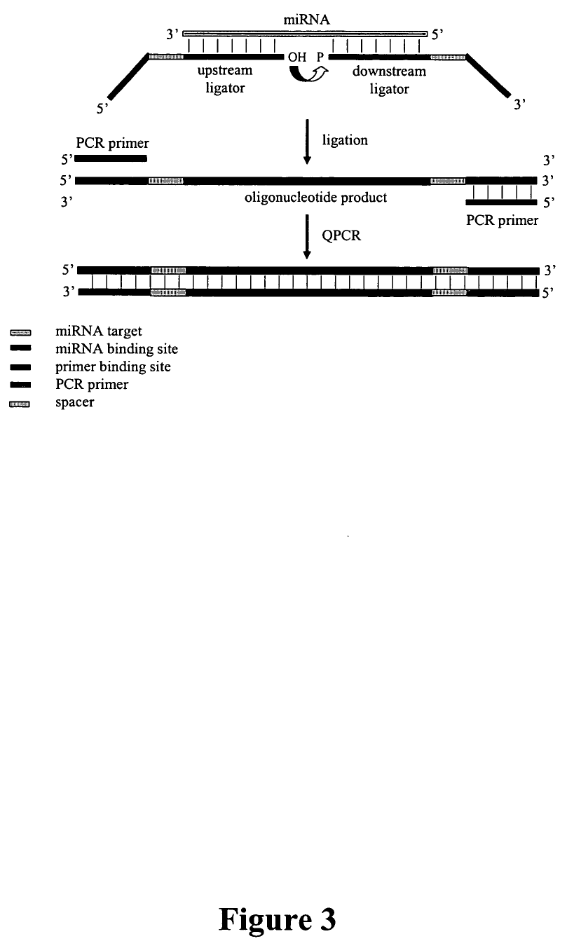

In preferred embodiments, the miRNA are isolated from cells, then detected by the ligation-QPCR assay of the invention (see FIG. 3, for exemplary schemes of the ligation-QPCR assay of the invention). The most commonly used method is to co-purify the miRNA with total RNA using a combination of acidified phenol and guanidine isothiocyanate using care not to remove the highly-soluble short RNA (see, for example, Pfeffer, S., Lagos-Quintana, M. & Tuschl, T. Cloning of Small RNA Molecules in Current Protocols in Molecular Biology (eds Ausubel, F. M. B. R. et al.) Ch. 26.4.1-26.4.18 (Wiley Interscience, New York, 2003). This method isolates total RNA, which comprises transfer RNA (tRNA), ribosomal RNA (rRNA), polyA messenger RNA (mRNA), short interfering RNA (siRNA), small nuclear RNA (snRNA), and microRNA (miRNA). If desired, the miRNA can be enriched from the total RNA by size selection using gel purification (Pfeffer, S., ibid).

Alternatively, the mirVana™ miRNA Isolation Kit (Ambion), which employs organic extraction followed by purification on a GFF using specialized binding and wash solutions, can be used to enrich for either long RNA or RNA of around less than 200 nucleotides. The resulting RNA preparation (less than about 200 nucleotides) is enriched for miRNAs, siRNAs, and/or snRNAs.

In addition, the Absolutely RNA® Miniprep Kit (Stratagene), which employs the traditional guanidine thiocyanate method and a silica-based matrix in a spin-cup format, is used to isolate total RNA comprising miRNA. Following lysis and homogenization of the tissue or cultured cells in lysis buffer, the sample is passed through a pre-filter by centrifugation to remove particulates and most of the DNA contamination. The clarified homogenate is mixed with ethanol and applied to the silica-based matrix RNA binding spin cup. After the RNA is washed, any bound DNA is hydrolyzed by DNase digestion. An additional wash removes the DNase, hydrolyzed DNA, and other impurities and the RNA is eluted from the spin cup with a low ionic strength buffer. The removal of DNA from the total RNA is a beneficial step as the genomic DNA includes the sequences that are transcribed and processed in miRNA. Complete removal of genomic DNA is desirable as its presence in the total RNA could lead to false or misleading results. While this method is not designed to isolate small RNA (<100 nucleotides), we have found that there is a significant amount of miRNA in the resulting RNA preparation. This is likely due to the interaction between a miRNA and its target mRNA resulting in their co-isolation.

In alternative embodiments, the miRNA are detected in a cell lysate without prior isolation or enrichment for small RNA, including miRNA. Such a method would allow for the ligation-QPCR assay and not allow for RNA degradation. Suitable methods include those described in Allawi, H. T., et al. Quantitation of microRNAs using a modified Invader assay. 2004. RNA. 10:113-1161 and Klebe, R. J., G. M. Grant, A. M. Grant, M. A. Garcia, T. A. Giambernardi, and G. P. Taylor. 1996. RT-PCR without RNA isolation. Biotechniques. 1996 December; 21(6): 1094-100.

In one exemplary embodiment, the method of the invention comprises providing two ligator oligonucleotides, providing a sample containing or suspected of containing an miRNA, providing two amplification oligonucleotide primers, combining the ligator oligonucleotides and sample to make a mixture, exposing the mixture to conditions that permit ligation of the two oligonucleotides to form a single ligation product, exposing the single ligation product, if present, to the two amplification primers, exposing the mixture to conditions that permit amplification of the single ligation product, if present, and detecting the presence or absence of amplification product.

The method of the invention can detect as few as 25,000 copies of an miRNA in a sample. This result compares very favorably against the known copy number of miRNA in various cells, which is reported to range from 1,000 to 500,000. Thus, the method of the invention can detect miRNA from as few as one cell. Typically, a sample will contain cell lysates or purified cell components from many cells (e.g., millions of cells); thus, the method of the invention is well suited for detection of miRNA from typical samples. Of course, parameters for detection may be adjusted to suit the individual practitioner's desires for speed and sensitivity. Therefore, while the method of the invention is capable of detected as few as 25,000 miRNA molecules in a cell sample, it may also be used to detect more, such as 50,000 molecules, 100,000 molecules, 250,000 molecules, 500,000 molecules, 1,000,000 molecules, or more. Likewise, while the method is capable of detecting an miRNA of interest in as few as one cell (or a lysate made therefrom), it can also detect an miRNA in a sample of many cells (or lysates therefrom), such as 100 cells, 1,000 cells, 10,000 cells, 50,000 cells, 100,000 cells, 500,000 cells, 1,000,000 cells, 10,000,000 cells, or more. As will be evident to those of skill in the art, the present method can detect any specific number of molecules of miRNA or cells within the range of these exemplary numbers, and thus, each particular number need not be stated.

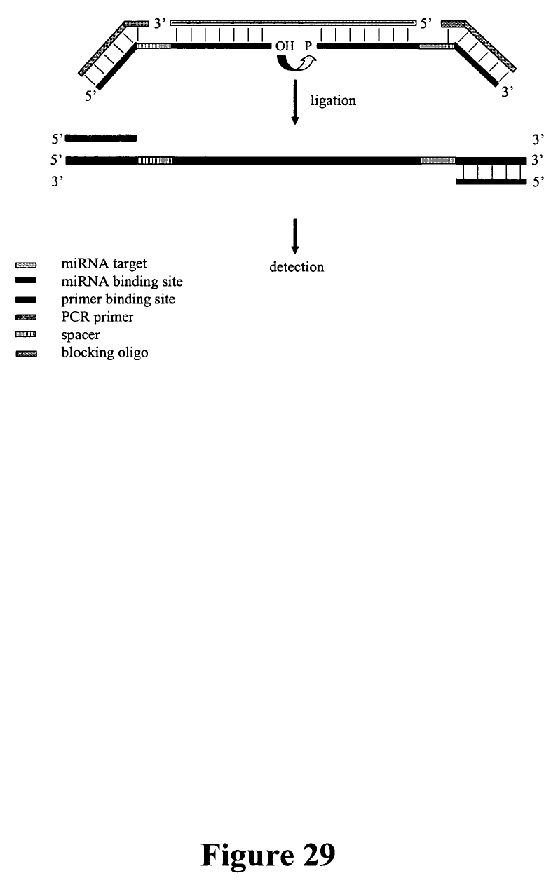

In yet another embodiment, blocking oligonucleotides complementary to the PCR priming site and spacer sequence, if present, (or the same as the PCR priming site and spacer sequence), are in the ligation reaction. See, for example, FIG. 29. The blocking oligonucleotides anneal to the PCR priming site and spacer sequence (or complements thereof) and reduce non-specific interactions that may occur between these sequences and those present in a sample. In this embodiment, the up and down ligators are essentially double-stranded except in the miRNA binding region. The blocking oligonucleotides may comprise modifications at the 3′ end to prevent ligation to or extension of the blocking oligonucleotide when annealed to a template. Suitable modifications include, but are not limited to, those that are commercially available: a 3′-amino nucleotide; a dideoxy nucleotide; a 3′-deoxy; a 2′-OH nucleotide; a reverse nucleotide, which could make the 3′ end of the oligo terminate in a 5′-OH; and 3′-alkyl-amino (C3-C10). The blocking oligonucleotides may comprise modifications at the 5′ end to prevent ligation to the blocking oligonucleotide. Suitable modifications include, but are not limited to, those that are commercially available: 5′-amino dT, 5′-OMe dT, and a 5′-amino modifier (C3-C10).

In a second aspect, nucleic acids are provided. The nucleic acids are generally nucleic acids that are useful in performing at least one embodiment of the method of the invention, or are created by at least one embodiment of the invention. The nucleic acids thus may be ligator oligonucleotides, amplification primers, ligation products (e.g., amplification templates), miRNA (for use as positive controls), and other nucleic acids that can serve as controls for one or more steps of the method.

The first class of nucleic acids provided by the invention are ligator oligonucleotides. Ligator oligonucleotides are oligonucleotides of any suitable length that can hybridize under appropriate conditions to a target miRNA. The ligators of the present invention comprise a region that is complementary, either completely or partially, to the target miRNA (miRNA complementary region) and can further comprise a PCR priming site (or a sequence complementary to a PCR priming site). In a preferred embodiment, the ligator also comprises a spacer region between the PCR priming site (or complement) and the miRNA complementary region.

Two ligators are designed for each target miRNA to anneal adjacent to each other when annealed to the target miRNA. The “up ligator” anneals to the 3′ portion of the miRNA and the “down ligator” anneals to the 5′ portion of the miRNA (see FIGS. 1 and 2, for example). The down ligator includes a phosphate (P— or [Phos]-) at the 5′ terminus (see, for example, FIGS. 1 and 2). The 5′ phosphate is beneficial for efficient ligation to the hydroxyl (—OH) at the 3′ terminus of the up ligator. In the presence of a ligase, the up and down ligators are ligated together when annealed to the target miRNA.

The miRNA complementary region is based on the nucleotide sequence of the target miRNA. miRNA ranging in length from 17 to 24 nucleotides in length have been identified (Griffiths-Jones S. The microRNA Registry. Nucleic Acids Res. 2004, 32, Database Issue, D109-D111). The point at which the ligators are joined may be varied and is dependent upon several factors including the relative melting temperatures (Tm) of the miRNA complementary region of the up and down ligators, the nucleotide preferences of the ligase that effect activity, the nucleotide preferences of the ligase that effect specificity, potential intra- and intermolecular interactions between the ligators, miRNA, and PCR primers, and a lack of homology to other published nucleotide sequences.

Typically, the ligator oligonucleotides hybridize to the target miRNA under stringent hybridization conditions (as used in the art). For example, hybridization of the ligator oligonucleotides may occur under the following conditions: ligation buffer—50 mM Tris-HCl, 4 mM DTT, 15 uM ATP, 4.5 mM MgCl₂, 0-25 mM NaCl, 30-55 mM KCl; ligase—4-10 U T4 DNA ligase; ligators—0.01-0.4 uM each ligator (each in the same amount or in varying ratios). Likewise, the conditions described in Example 1, below, are suitable. In certain embodiments, the ligator oligonucleotides hybridize under hybridization conditions that approach or are only slightly lower than conditions that disfavor hybridization of the ligator oligonucleotides and the target miRNA sequences. Because of the high secondary structure that can be present in pri-miRNA and pre-miRNA, it can be important to adjust hybridization conditions to minimize the amount of self-hybridization of the miRNA during the hybridization period. Likewise, as discussed below, in embodiments the ligator oligonucleotides are designed to contain secondary structures. Thus, it can be desirable to set the hybridization conditions to those that are only slightly lower than the conditions that disfavor hybridization to ensure that both the target miRNA and the ligator oligonucleotides are in extended forms suitable for hybridization to each other. Furthermore, in view of the short length of the miRNA and the region of hybridization (9-15 nucleotides), it can be important to raise the stringency of the hybridization conditions to limit the amount of hybridization of the ligator oligonucleotides to non-target nucleic acid sequences.

Various methods are available to estimate the melting temperature (Tm) of the annealed up ligator and the target miRNA and the annealed down ligator and the target miRNA. The Tm is the temperature at which 50% of the nucleotide sequence and its perfect complement are in duplex. These methods apply to estimating the Tm of DNA:DNA hybrids, of RNA:RNA hybrids, and of DNA:RNA hybrids. The methods of estimating the Tm for DNA:DNA hybrids range from the crude estimation given by 2° C. for each A:T and 4° C. for each G:C (Wallace, R. B., J. Shaffer, R. R. Murphy, J. Bonner, T. Hirose, and K. Itakura, 1979. Nucleic Acids Res. 6, 3543) to the nearest neighbor method used by Mfold (Zuker, M. 2003. Mfold web server for nucleic acid folding and hybridization prediction. Nucleic Acids Res. 31(13): 3406-3415 and Mathews, D. H., J. Sabina, M. Zuker and D. H. Turner. 1999. Expanded Sequence Dependence of Thermodynamic Parameters Improves Prediction of RNA Secondary Structure. J. Mol. Biol. 288, 911-940). Mfold is based on the effect of the nucleotide sequence and is considered to be the most accurate method of estimating Tm. Mfold allows the user to define some of the variables of the ligation conditions, including temperature, salt concentration, and magnesium concentration.

More recently, methods have been developed to estimate the Tm of DNA:RNA hybrids for use in anti-sense technology (Sugimoto, N., S. Nakano, M. Katoh, A. Matsumura, H. Nakamuta, T. Ohmichi, M. Yoneyama, and M. Sasaki. 1995. Thermodynamic parameters to predict stability of RNA/DNA hybrid duplexes. Biochemistry. 34(35):12,211-12,116; Gray, D. M., 1997. Derivation of nearest-neighbor properties from data on nucleic acid oligomers. II. Thermodynamic parameters of DNA:RNA hybrids and DNA duplexes. Biopolymers. 42(7):795-810) and Le Novere, N., 2001. MELTING, computing the melting temperature of nucleic acid duplex. Bioinformatics. 17(12):1226-1227). When the stability of RNA:RNA, RNA:DNA, and DNA:DNA were compared, the most stable duplex was RNA:RNA. Whether the RNA:DNA or DNA:DNA duplex was more stable was dependent upon the nucleotide sequence. This sequence dependence is considered when calculating the Tm of DNA:RNA based using the nearest-neighbor method (http://bioweb.pasteur.fr/seqanal/interfaces/melting.html). The nearest-neighbor equation for DNA and RNA-based oligos is: (1) Tm=(1000ΔH/A+ΔS+Rln (C/4))−273.15+16.6 log[Na+] (For DNA see: Breslauer, K, J., R. Frank, H. Blocker, L. A. Marky, 1986. Proc. Natl. Acad. Sci. USA 83:3746-3750 and for RNA see: Freier, S. M., R. Kierzek, J. A. Jaeger, N. Sugimoto, M. H. Caruthers, T. Neilson, D. H. Turner, 1986. Proc. Natl. Acad. Sci. 83:9373-9377) ΔH (Kcal/mol) is the sum of the nearest-neighbor enthalpy changes for duplexes. A is a constant containing corrections for helix initiation. ΔS is the sum of the nearest-neighbor entropy changes. R is the Gas Constant (1.99 cal K-1 mol-1), and C is the concentration of the oligonucleotides. Exemplary ΔH and ΔS values for nearest neighbor interactions of DNA and RNA are shown in Table 1. In many cases this equation gives values that are no more than 5° C. from the empirical value. It is good to note that this equation includes a factor to adjust for salt concentration.

TABLE 1

Thermodynamic parameters for nearest-
neighbor melting temperature formula

| | DNA | | RNA | |
|---|---|---|---|---|
| Interaction | ΔH | ΔS | ΔH | ΔS |

| | | | | |
|---|---|---|---|---|
| AA/TT | −9.1 | −24.0 | −6.6 | −18.4 |
| AT/TA | −8.6 | −23.9 | −5.7 | −15.5 |
| TA/AT | −6.0 | −16.9 | −8.1 | −22.6 |
| CA/GT | −5.8 | −12.9 | −10.5 | −27.8 |
| GT/CA | −6.5 | −17.3 | −10.2 | −26.2 |
| CT/GA | −7.8 | −20.8 | −7.6 | −19.2 |
| GA/CT | −5.6 | −13.5 | −13.3 | −35.5 |
| CG/GC | −11.9 | −27.8 | −8.0 | −19.4 |
| GC/CG | −11.1 | −26.7 | −14.2 | −34.9 |
| GG/CC | −11.0 | −26.6 | −12.2 | −29.7 |
| | 0.0 | −10.8 | 0.0 | −10.8 |

While these methods are useful in estimating the Tm of duplexes, a method to empirically determine the Tm of the duplexes of this invention is also useful. A common method is to use a temperature-controlled cell in a UV spectrophotometer and measure absorbance over a range of temperatures. When temperature is plotted vs. absorbance, an S-shaped curve with two plateaus is observed. The temperature reading halfway the plateaus corresponds to the Tm. Alternatively, a thermocycler such as the MX3000P with samples comprising a nucleic acid dye that binds double-stranded nucleic acid with higher affinity than single-stranded nucleic acid, such as SYBR Green (Molecular Probes), is used to generate the plot with temperature vs. absorbance.

In this example, the Tm were calculated using the Schepartz Lab Biopolymer Calculator available at http://paris.chem.yale.edu/extinct.html (DNA:DNA) (Table 2).

TABLE 2

Nucleotide sequence of target miRNA and the
effect of the ligation position on the Tm
of portion of the up and down ligators when
annealed to the miRNA

| | miRNA Nucleotide | miRNA | SEQ |
|---|---|---|---|
| | Sequence (5′-3′) | Length | ID |
| Name | and Relative Tm | (nt) | NO: |
| Hsa-let-7d | 28° C. [symbol] \| → 32° C. | 21 | |
| | AGAGGUAGUAGGUUGCAUAGU | | |
| | 26° C. [symbol] \| → 34° C. | | |
| Hsa-miR-15a | 32° C. [symbol] \| → 30° C. | 22 | |
| | UAGCAGCACAUAAUGGUUUGUG | | |
| Hsa-miR-16 | 34° C. [symbol] \| → 30° C. | 22 | |
| | UAGCAGCACGUAAAUAUUGGCG | | |
| | 32° C. [symbol] \| → 32° C. | | |
| Hsa-miR-125b | 36° C. [symbol] \| → 30° C. | 22 | |
| | UCCCUGAGACCCUAACUUGUGA | | |
| | 32° C. [symbol] \| → 34° C. | | |

Alternatively, the ligator oligonucleotides can be characterized in terms of their percent identity to the miRNA target sequences. In general, the ligator oligonucleotides show at least 70% sequence identity with the target miRNA over a stretch of 9-15 nucleotides. Thus, over any chosen 9-15 nucleotide sequence, a ligator oligonucleotide can show precisely or about 70% or greater identity, 75% or greater identity, 80% or greater identity, 90% or greater identity, 91% or greater identity, 92% or greater identity, 93% or greater identity, 94% or greater identity, 95% or greater identity, 96% or greater identity, 97% or greater identity, 98% or greater identity, 99% identity, or greater than 99% identity, such as 100% identity to a 9-13 nucleotide sequence of a target miRNA.

It is to be noted at this point that each value stated in this disclosure is not, unless otherwise stated, meant to be precisely limited to that particular value. Rather, it is meant to indicate the stated value and any statistically insignificant values surrounding it. As a general rule, unless otherwise noted or evident from the context of the disclosure, each value includes an inherent range of 5% above and below the stated value. At times, this concept is captured by use of the term “about”. However, the absence of the term “about” in reference to a number does not indicate that the value is meant to mean “precisely” or “exactly”. Rather, it is only when the terms “precisely” or “exactly” (or another term clearly indicating precision) are used is one to understand that a value is so limited. In such cases, the stated value will be defined by the normal rules of rounding based on significant digits recited. Thus, for example, recitation of the value “100” means any whole or fractional value between 95 and 105, whereas recitation of the value “exactly 100” means 99.5 to 100.4.

In view of the fact that the ligator oligonucleotides may comprise a sequence that can hybridize with a target sequence on an miRNA of interest, but that might not show 100% identity with that target sequence, it is evident that the ligator oligonucleotides can hybridize with sequences of other miRNA, such as miRNA that are related to the miRNA of interest. Accordingly, the ligator oligonucleotides can be used to identify unknown miRNA that have a certain level of sequence identity with a known miRNA. Likewise, the ligator oligonucleotide sequences and/or the hybridization and ligation conditions can be adjusted such that the ligator oligonucleotides bind to and detect two or more members of the same miRNA family. In this way, a general understanding of the extent to which family members are present in a sample can be gained. In such a situation, if the practitioner desires to identify the individual members of the family that have been detected, hybridization and ligation conditions may be adjusted, or the sequence of the ligator oligonucleotides may be altered to raise the specificity. In doing so, one or both of the ligator oligonucleotide sequences can be altered, for example, based on the known sequence of an miRNA.

In addition, it is contemplated that the various changes to the miRNA binding region of the ligator oligonucleotides will be made in the knowledge that certain changes will have more profound effects on binding to target miRNA than others. Numerous algorithms are publicly available and widely used to estimate the effect of various changes in a given sequence on its ability to hybridize to a target sequence. Thus, for example, changes that result in mismatches at or near the ligation site are often destabilizing and decrease the efficiency of hybridization and ligation. Likewise, multiple mismatching nucleotides adjacent to each other and at internal bases generally tend to destabilize hybridization to a greater extent than if the same number of mismatches are distributed about the sequence or are at the terminus that is not directly involved in ligation. Where a practitioner desires to design a ligator sequence that will detect multiple members of an miRNA family, or miRNA species that show certain levels of identity to a known miRNA, these well-known considerations will often be taken into account.

In addition, it should be recognized that different ligases have different levels of tolerance for base composition and/or mismatches at, near, or distal to the site of ligation. Such tolerances have been identified and characterized in the art. Accordingly, the practitioner may select the ligase to be used in conjunction with the base composition of one or both of the ligator oligonucleotides to achieve suitable or desired levels of ligation. The practitioner may also select the ligase in conjunction with the number, type, and/or location of mismatches in one or both of the ligator oligonucleotides to achieve suitable or desired levels of ligation or different levels of specificity for a particular miRNA or group of miRNA with related sequences.

As discussed above, the method of the invention relies on the target miRNA bringing two ligator oligonucleotides into close enough proximity such that the two can be ligated to form a single ligation product. In view of this concept, ligator oligonucleotides are typically designed in pairs such that both will hybridize to the target miRNA in a way that places the 5′ end of one ligator oligonucleotide adjacent to the 3′ end of the other ligator oligonucleotide. (See, for example, FIG. 1). Accordingly, these portions of the ligator oligonucleotides contain sequences that are complementary (within the percent identity ranges discussed above) to sequences in the miRNA. The remaining portions of the ligator oligonucleotides may be designed based on numerous other considerations, some of which will be discussed immediately below, some of which will be apparent to those of skill in the art, and some of which may be selected by the practitioner based on particular desires for particular assays.

In embodiments, the two termini of the ligator oligonucleotides to be used in an assay (that is the 3′ terminus of one and the 5′ terminus of the other) are designed to contain nucleotides that are preferred for one or more pre-selected ligases. For example, the ligation point may be engineered to include preferred nucleotides for T4 DNA ligase by adjusting the size of each ligator oligonucleotide. For example, for an miRNA of 25 nucleotides in length, one ligator oligonucleotide may have a hybridization sequence of 15 nucleotides while the other has a hybridization sequence of 9 nucleotides in order to generate a ligation point that is optimal for T4 DNA ligase.

While exemplary ligators of this invention were designed to ligate when adjacently annealed to the target miRNA, it has been found that ligation of the ligators occurs to some extent in the absence of target miRNA and in the presence and absence of Torulla yeast RNA. Torulla yeast RNA was used in the experiments disclosed herein as a neutral source of RNA because it is derived from Torulla, a budding yeast, and miRNA have not been described in budding yeast. Template-independent ligation was previously described for T4 DNA and Escherichia DNA ligases. (Barringer, K. J., L. Orgel. G. Wahl, and T. R. Gingeras 1990. Blunt-end and single-strand ligations by Escherichia coli ligase: influence on an in vitro amplification scheme. Gene. 89(1):117-122). Although the method of the invention functions well with the background levels of non-template mediated ligation, methods to reduce or eliminate this template-independent ligation were devised, and include the use of a different ligase, different ligation conditions, and/or the use of additives in the ligation reaction. Such additives include Perfect Match® PCR Enhancer (Stratagene). Additionally, experiments that identify those ligator sequences that are less likely to participate in template-independent ligation are also contemplated. Thus, those sequences would be considered during the ligator design process.

Intra- and inter-molecular interactions within and between the individual up and down ligators, the ligation product of the up and down ligators, the miRNA template, and the PCR primers can result in undesirable side reactions instead of or in addition to ligation of the up and down ligators. Intra-molecular interactions are estimated using programs such as Mfold (version 3.1) (Zuker, M., above), which uses the nearest neighbor energy rules to assign free energies to loops rather than to base pairs. Intermolecular interactions are estimated using common primer design programs such as Primer Designer 4.0 (Sci Ed Central). One of skill in the art can select criteria based on the level of specificity desired.

In silico nucleotide sequence comparisons between potentially useful sequences and published human genomic DNA can be made using BLAST (Altschul, S. F., Gish, W., Miller, W., Myers, E. W. and Lipman, D. J. 1990. Basic local alignment search tool. J. Mol. Biol. 215:403-410). While this method is useful, it has been found that a QPCR using the potentially useful sequence and genomic DNA or cDNA from the organism of interest as template (for example, human genomic DNA) be performed to validate the in silico findings.

One feature of the present invention is the ability to rapidly and easily detect a small molecule, such as a 18-25 nucleotide miRNA. This feature is achieved by ligating two relatively large ligator oligonucleotides together, using the target miRNA as a template for their juxtaposition. The resulting ligation product is large, relative to the target miRNA, and can be detected easily and/or rapidly by numerous techniques. The size of the ligation product can be any size selected by the practitioner, but will typically be in the range of 50-500 nucleotides. For example, the ligation product can be 50-100 nucleotides in length, 50-150 nucleotides in length, or 50-200 nucleotides in length. It can also be 75-125 nucleotides in length, 75-150 nucleotides in length, 74-100 nucleotides in length, 90-130 nucleotides in length, or 100-140 nucleotides in length. Any specific nucleotide length within these ranges is a suitable length, and thus each particular value need not be recited herein. Other suitable lengths can be chosen to achieve a ligation product, and such lengths are encompassed by this invention. Techniques for detection of ligation products can be chosen by those of skill in the art based on numerous considerations, all of which are well within the skill level of those of skill in the art. For example, relatively long ligation products may be amenable to detection using standard gel electrophoresis and staining techniques. On the other hand, ligation products of 150 bases or less (e.g., 75-150 nucleotides) may be efficiently detected using QPCR and SYBR Green staining. In general, either the length of the ligation product will be engineered based on a desired detection technique, or a desired detection technique will be chosen based, at least in part, on the detection method desired. Because numerous different detection techniques are now commonplace, there is no particular preference for one length of ligation product over any other.

In addition to the miRNA binding site, the ligator oligonucleotides thus comprise non-binding nucleotides that provide length, and optionally other features. These non-miRNA binding nucleotides can be randomly included in the ligator oligonucleotides or the sequences of such nucleotides can be designed for particular purposes. In embodiments, the non-binding nucleotides are specifically included in one or more particular sequences or in relative amounts of adenine, guanine, cytosine, and thymine (or uracil, depending on the desire of the practitioner) so as to provide binding sites for one or more short oligonucleotides, such as amplification primers or detection probes. Although the amplification primer binding sites will typically be located at or near the ends of the ligator probes that will form the 3′ and 5′ ends of the ligation product (so as to maximize the length of amplification product), they may be placed at any suitable point along the ligator oligonucleotide sequence. In embodiments where amplification will be performed after ligation, because the ligation product will be the template for amplification, it may be desirable to engineer amplification primer binding sites that have similar melting temperatures to each other to facilitate accurate and robust amplification.

In embodiments where a PCR primer binding site (or a sequence complementary to a PCR primer binding site) is included in the ligator oligonucleotide sequence, the PCR priming site typically allows for annealing of the complementary PCR primer during QPCR to allow for synthesis of additional copies of the ligated ligators (i.e., the ligation product). The PCR priming sites and corresponding PCR primers can be designed according to the guidelines given in the manual for the Brilliant® SYBR® Green QPCR Master Mix (Stratagene). In this example, randomly generated sequences are analyzed for 1) intermolecular interactions using primer design software (Primer Designer 4.0), 2) intra-molecular interactions (Mfold), and 3) homology to the human genome (BLAST). While this method is useful in identifying and eliminating PCR primer sequences with significant homology to published nucleotide sequences, a QPCR using genomic DNA from a commercial source (BD Biosciences) to verify that the PCR primers did not generate PCR products in the absence of ligated ligators was performed as described below.

The ligator oligonucleotides may comprise, in addition to miRNA binding sequences, sequences that do not provide any sequence-specific function. These are referred to herein at various times as “spacer” or “linker” sequences. These spacer or linker sequences mainly provide length for the entire ligation product, and thus can vary widely is length from one oligonucleotide to the next, including between two oligonucleotides that are designed to be used to identify a single particular miRNA. In general, the linker or spacer is of a sufficient length to yield a final ligation product of 74 nucleotides or greater, taking into account all other sequences present in both ligator oligonucleotides that are to participate in the miRNA-mediated ligation. As a general rule, design of the linker sequences should follow the general considerations for PCR primers (e.g., no significant homology to sequences in the genome of the organism being studied, no significant secondary structure or structures that can be formed between two ligator oligonucleotides).

In embodiments, the ligators thus comprise a spacer sequence to increase the length of the ligated ligators. Among the advantages provided by the spacer, the increase in length can provide an efficient template for the QPCR when using SYBR® Green (Molecular Probes) for detection. In embodiments, randomly generated sequences can be added to the ligators between the PCR priming site and the miRNA annealing sequence. If desired, these can be analyzed for 1) intermolecular interactions using primer design software (Primer Designer 4.0), 2) intra-molecular interactions (Mfold), and 3) homology to the human genome (BLAST). They can also be analyzed for their respective Tm and the identity and/or position of various nucleotides altered to obtain oligonucleotides with suitable characteristics.

Other nucleotide sequences that can be provided on the ligator oligonucleotides include, but are not limited to, sequences for binding of detection moieties (e.g., TaqMan binding sequences), sequences for sequence-specific capture probes, sequences for additional amplification probes (on one or both of the ligator oligonucleotides to be used for ligation), restriction endonuclease recognition and/or cleavage sites, and sequences that are known to be recognition or modification sites for nucleic acid modifying enzymes (e.g., methylation sites). The addition of such sequences permit any number of additional pieces of information to be generated during an assay. For example, addition of TaqMan binding sequences permits multiplexing. Thus, in an embodiment, one or both of the ligators include a probe-binding region (see FIG. 3) to allow for annealing of a hydrolysis probe having a fluorophore, which can be located at the 5′ end of the probe, and a quencher that is either internal or located at the 3′ end of the probe (see, for example, Higuchi, R., Fockler, C., Dollinger, G. and Watson, R. Kinetic PCR analysis: real-time monitoring of DNA amplification reactions. 1993. Biotechnology (NY). 11(9):1026-30 and Holland, P. M., Abramson, R. D., Watson, R. and Gelfand, D. H. Detection of specific polymerase chain reaction product by utilizing the 5′ - - - 3′ exonuclease activity of Thermus aquaticus DNA polymerase. 1991. Proc. Natl. Acad. Sci. USA 88(16):7276-80). When a hydrolysis probe is used for detection of target miRNA, FullVelocity™ QPCR Master Mix (Stratagene) can be used.

The up and/or down ligators may include nucleoside analogues to improve annealing specificity and/or ligation efficiency. As previously stated, many of the miRNA belong to a family of miRNA based on sequence similarities. For example, one of the miRNA specifically examined in the present invention, let-7d, is a member of the let-7 family. The let-7 family has 10 members with high sequence similarities (Table 3). As can be seen in Table 3, the high sequence similarity is primarily on the 5′ portion of the miRNA. An embodiment which increases sequence specificity therefore focuses on the 5′ portion of the miRNA.

TABLE 3

Nucleotide Sequence of Let-7 and
related miRNA family

| members | Nucleotide Sequence | |
|---|---|---|
| miRNA | (5′ to 3′) | SEQ ID NO: |
| let-7a-1 | TGAGGTAGTAGGTTGTATAGTT | |
| let-7f-1 | TGAGGTAGTAGATTGTATAGTT | |
| let-7i | TGAGGTAGTAGTTTGT GCT | |
| let-7h | TGAGGTAGTAGTGTGTACAGTT | |
| let-7g | TGAGGTAGTAGTTTGTACAGTA | |
| let-7d | AGAGGTAGTAGGTTGCATAGT | |
| let-7e | TGAGGTAGGAGGTTGTATAGT | |
| let-7c | TGAGGTAGTAGGTTGTATGGTT | |
| let-7b | TGAGGTAGTAGGTTGTGTGGTT | |
| miR-98 | TGAGGTAGTAAGTTGTATTGTT | |
| miR-84 | TGAGGTAGTATGTAATATTGTA | |

In embodiments, the linker region and primer binding region are engineered as standard or “universal” sequences that can be used as individual units or a single unit to be shuffled with different miRNA binding sequences that are specific for different miRNA. In this way, a standardized expression and detection system can be developed that is consistent from one miRNA to another.

In certain embodiments, the ligator oligonucleotides are designed to have no significant secondary structure (as determined by Zucker's Mfold program). In certain other embodiments, the ligator oligonucleotides are designed to have secondary structure at room temperature and moderate salt conditions. In view of this design option, it is evident that some non-miRNA binding sequences of certain ligator oligonucleotides will be selected to enable secondary structures (e.g., hairpin loops) to form. Such structures can increase hybridization specificity. It is envisioned that such secondary structures will have melting temperatures lower than the melting temperatures of the miRNA and each ligator oligonucleotide, lower than the melting temperatures of the ligator oligonucleotides and one or more amplification primers, or both. Preferably, both ligator oligonucleotides to be used to detect a target miRNA will have melting temperatures that are precisely or about the same. In embodiments, only one of a pair of ligator oligonucleotides will have secondary structure, such as a hairpin structure. In other embodiments, both ligator oligonucleotides will have secondary structure.

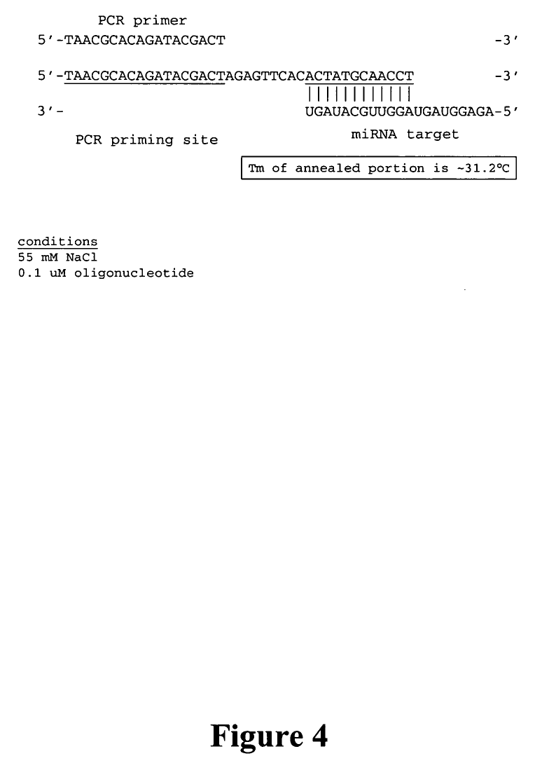

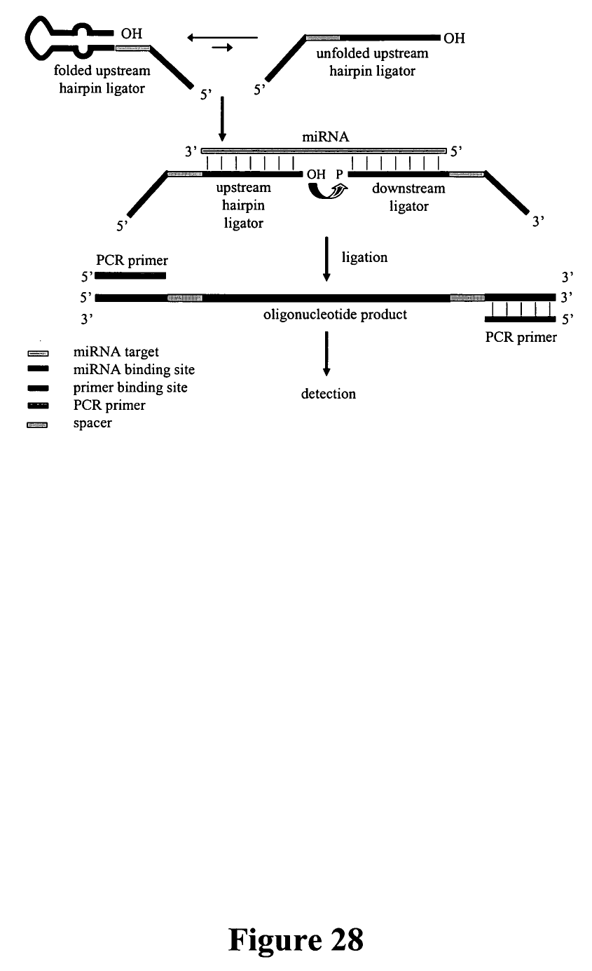

Accordingly, in embodiments, the ligators include a hairpin at the 3′ end of the up ligator and/or at the 5′ end of the down ligator. The hairpin introduces partial self-complementarity into the ligator and allows the 3′ or 5′ end of the up or down ligator, respectively, to fold back on itself to form a hairpin (see, for example, FIGS. 4-19 and 28). Either one or both of the up and down ligators may have a hairpin. The hairpin sequences may be between the PCR priming sites and the miRNA complementary region or contained, either partially or completely, within these regions. The hairpin sequences may be within the spacer region or in addition to the spacer region. The hairpin sequences may also be 5′ of the PCR priming sites or 3′ of the PCR priming sites. In certain embodiments, ligator oligonucleotides are designed such that a hairpin structure is present in each, and where the complementary portion that forms part of the stem of the hairpin is 5′ of a PCR priming site in the down ligator, and 3′ of a PCR priming site in the up ligator. The hairpins would essentially form a circle with the ends forming a small stem. After PCR, the complementary sequences forming the stem would not be present, as some of the bases would not have been amplified during PCR.

The hairpin can comprise a stem and loop structure wherein the stem structure is partially base paired with the miRNA annealing portion of the ligator. The hairpin is often designed to have a higher binding constant when bound to the miRNA than when binding to the ligator. A higher binding constant refers to having more unfolded hairpin molecules bound to the miRNA than folded hairpin molecules under the same conditions. Use of the hairpin ligator can increase specificity during the annealing reaction by reducing or eliminating binding to non-target miRNA and/or decrease ligation of the up and down ligators in the absence of template (template-independent ligation).

The relative Tm of the hairpin when the ligator is folded upon itself and when the unfolded hairpin is base paired with the target miRNA can be an important criteria when designing a ligator hairpin. Thus, it should be considered for each ligator oligo designed. As discussed above, selection of appropriate sequences can be performed using well-known and widely used computer programs, and may easily be tested if desired.

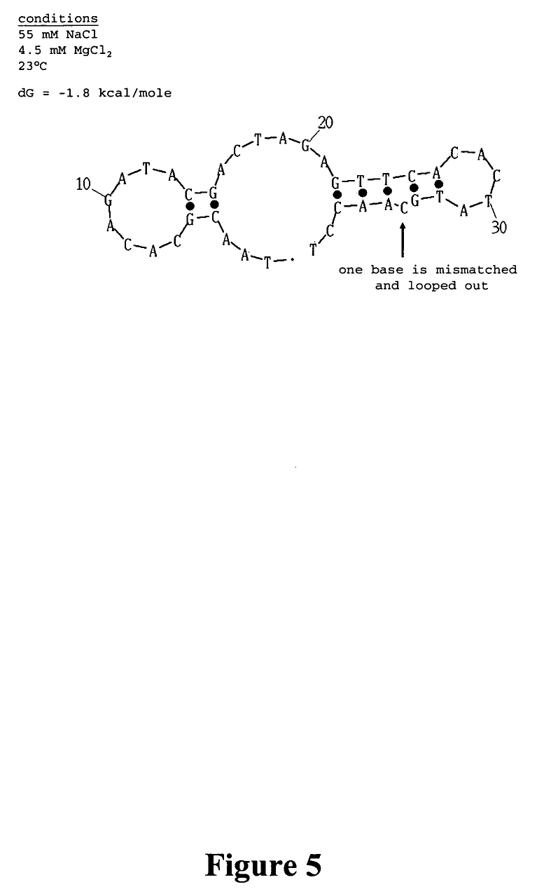

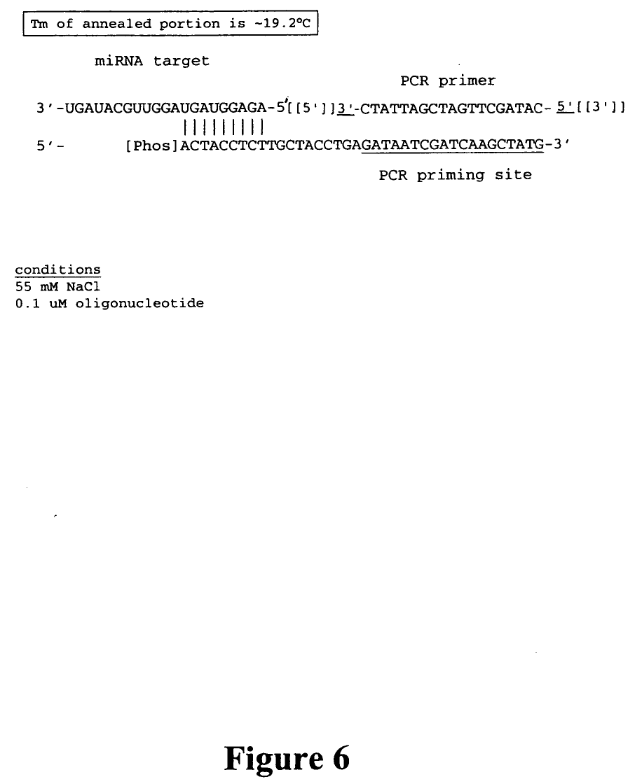

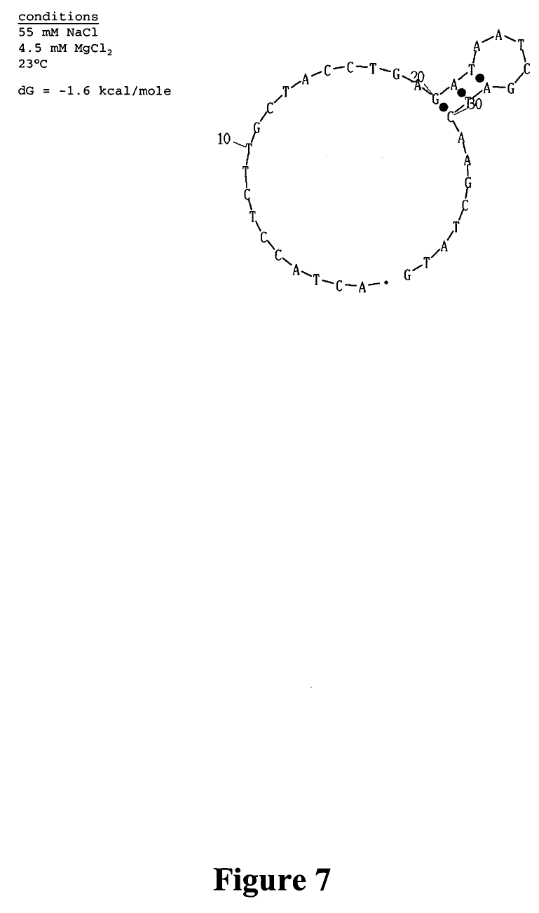

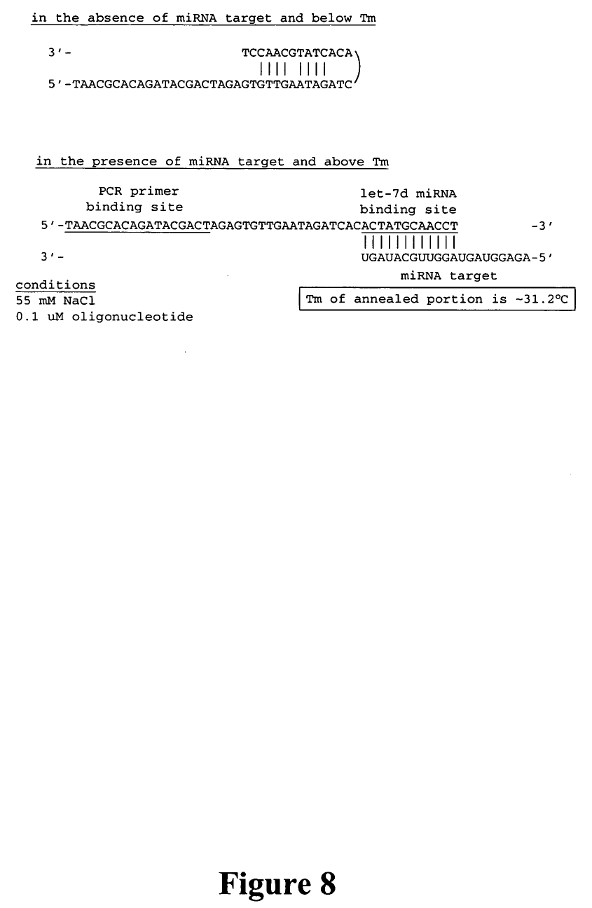

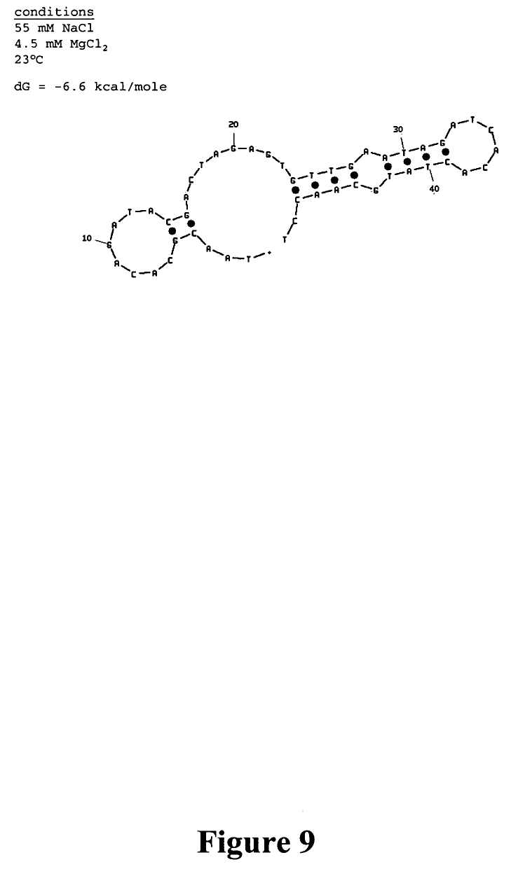

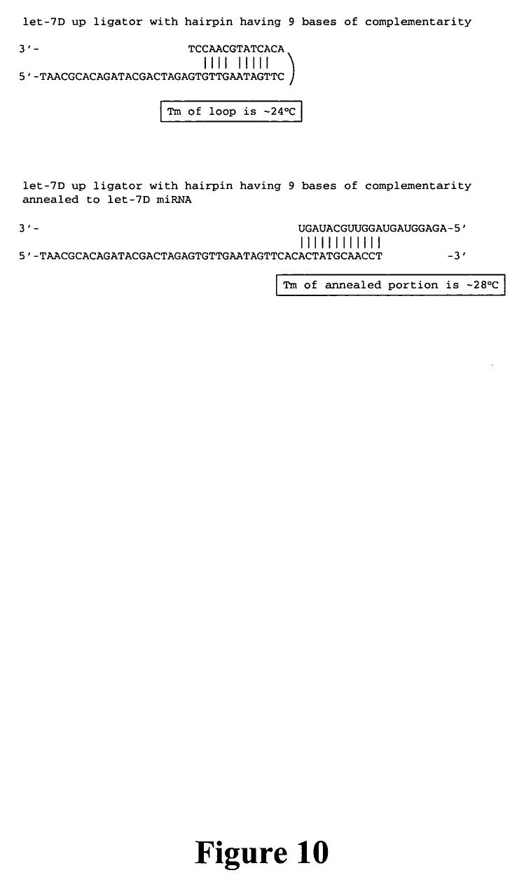

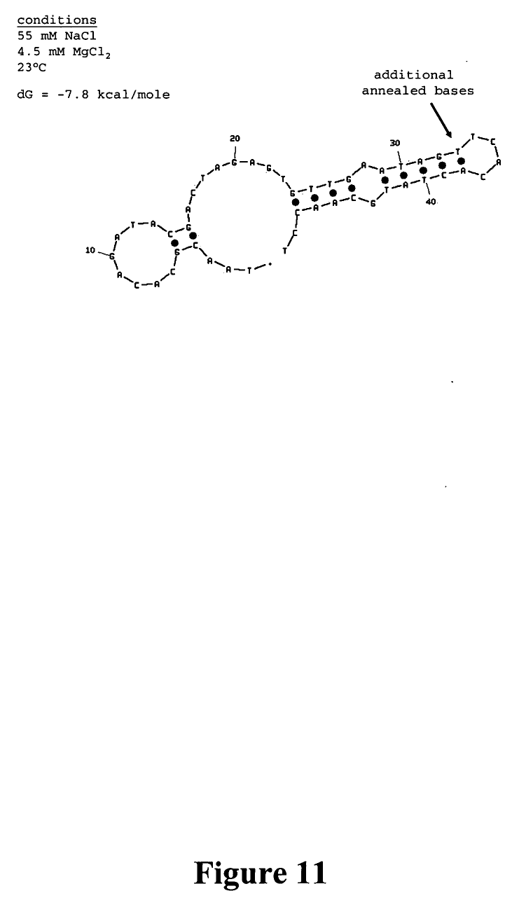

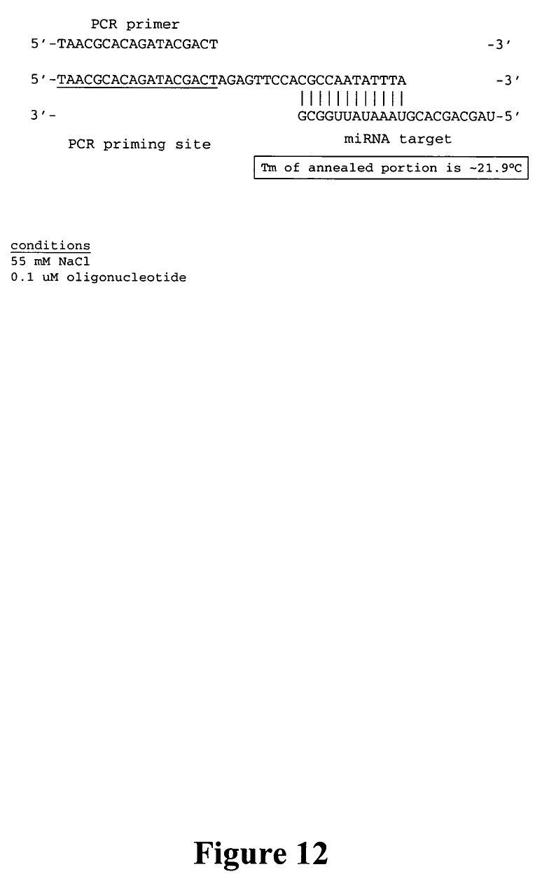

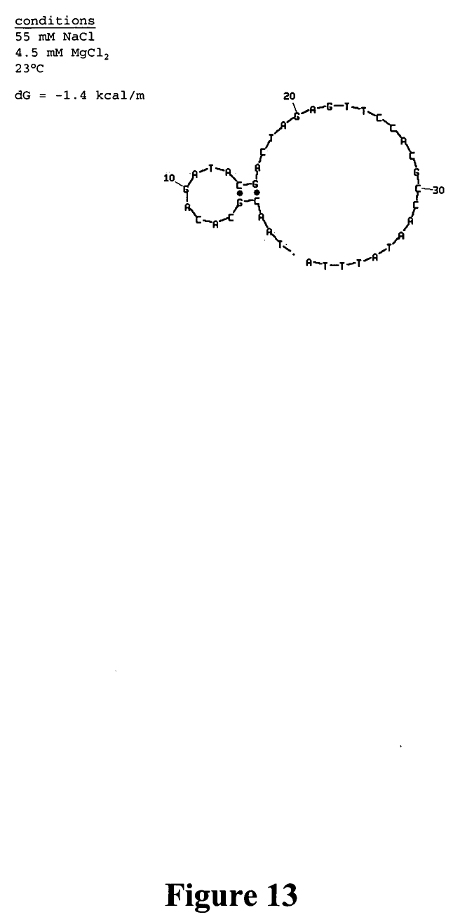

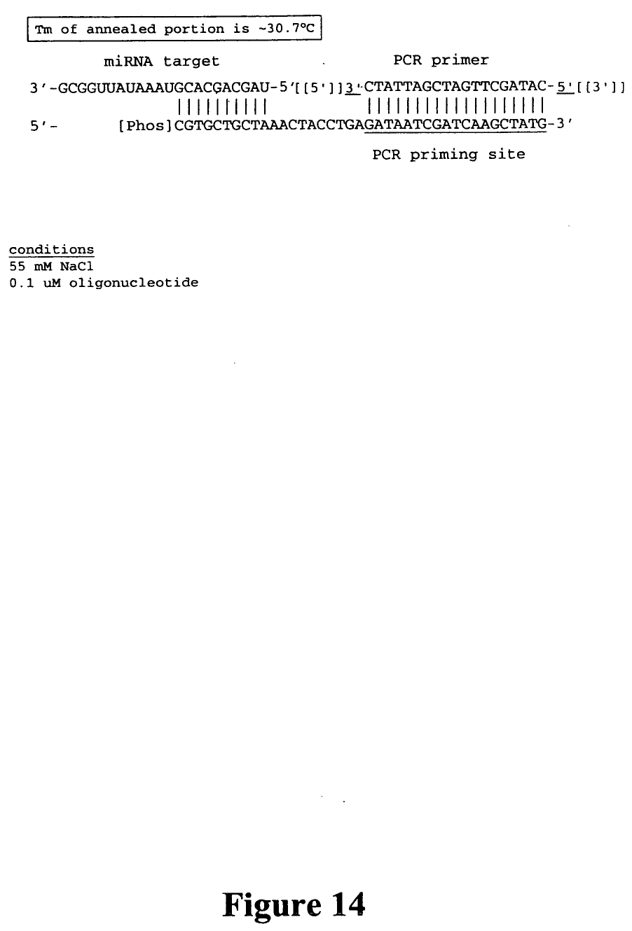

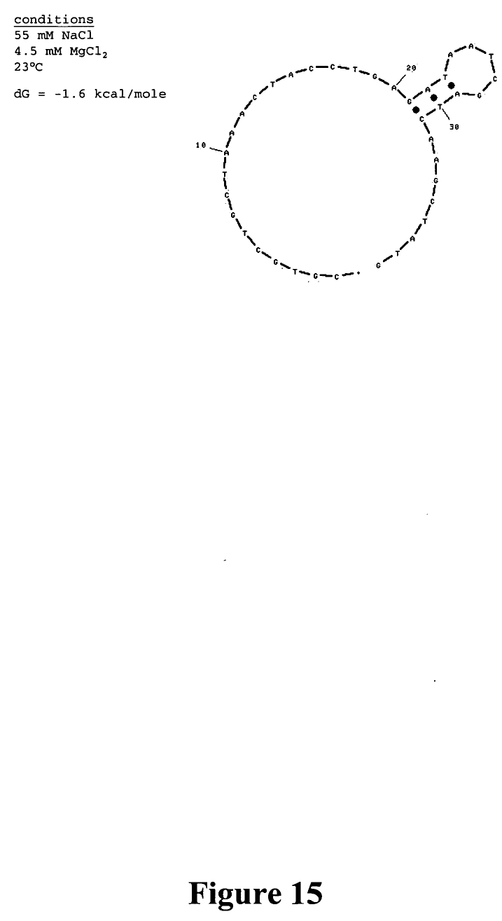

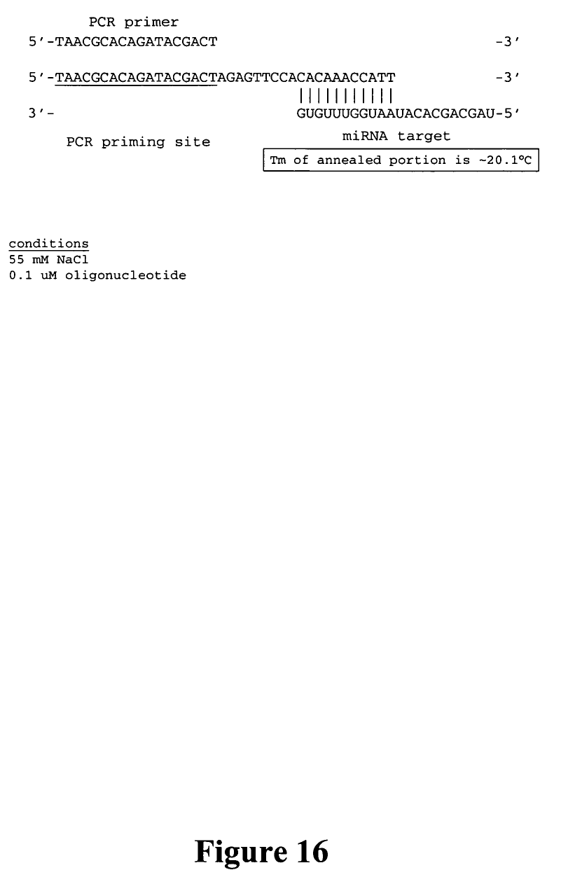

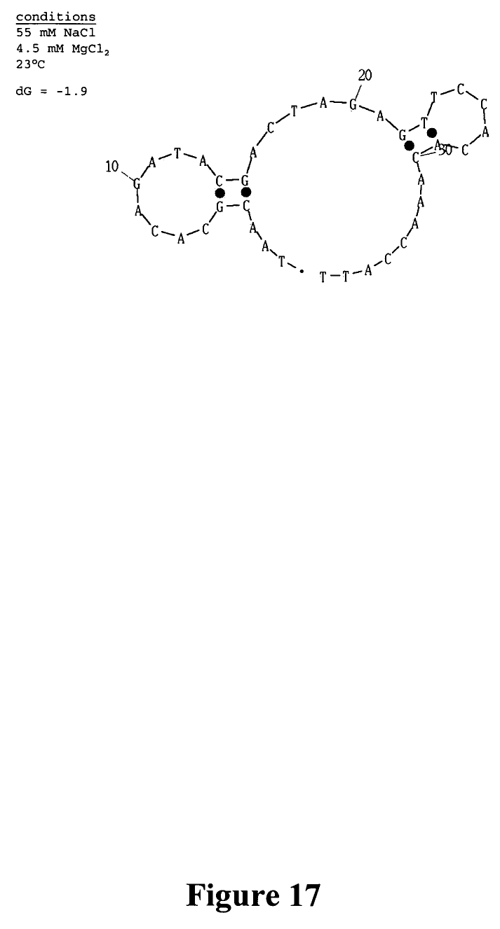

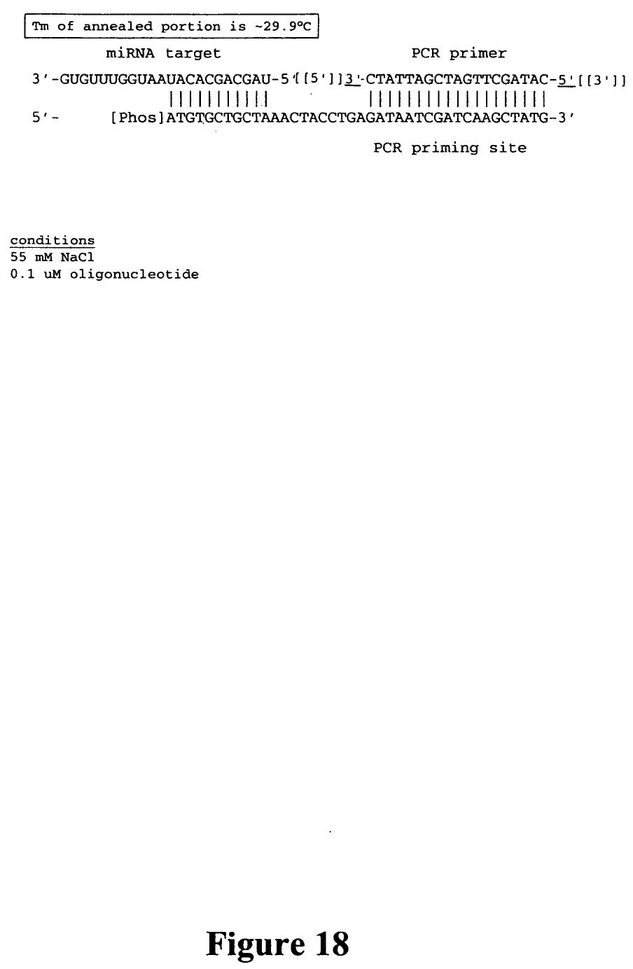

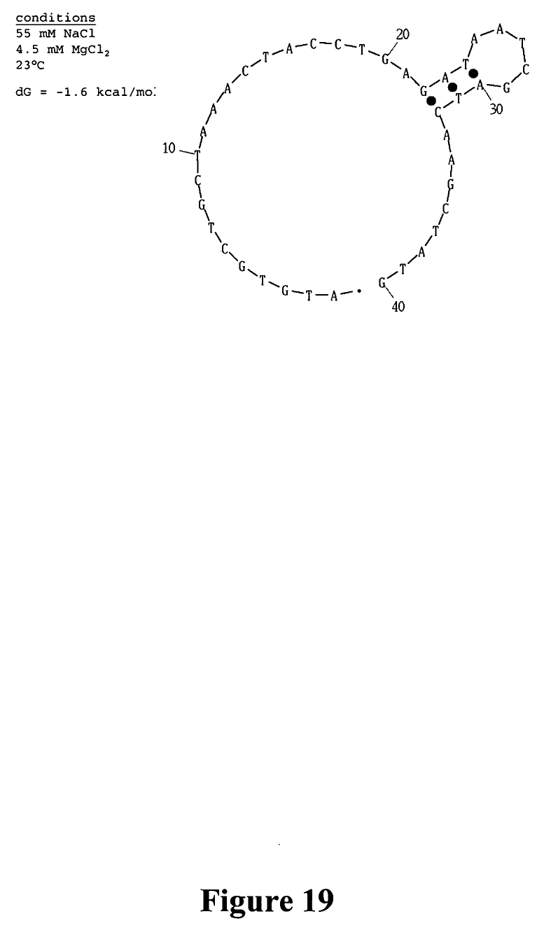

Examples of different ligator sequences are presented in FIGS. 4-19. FIG. 5 shows one embodiment of an up ligator for the let-7d miRNA, which has been designed to have 7 total base pairs, forming two separate hairpin-loop structures. FIGS. 6 and 7 depict one embodiment of a down ligator for the let-7d miRNA, having a single hairpin-loop structure defined by a three base pair region of complementarity. FIGS. 8 and 9 show another embodiment of an up ligator for the let-7d miRNA, designed to have two hairpin-loop structures, one with a two base pair region of complementarity and the other with an 8 base pair region of complementarity. FIGS. 10 and 11 show yet another embodiment of an up ligator for the let-7d miRNA. In this embodiment, the ligator oligonucleotide has a region of 9 bases of self-complementarity. FIGS. 12 and 13 depict another embodiment of the invention, in which an exemplary miR-16 up ligator has been engineered to include a two base pair region of self-complementarity. In another embodiment, depicted in FIGS. 14 and 15, an miR-16 down ligator having a region of three bases of complementarity is provided. In yet another embodiment, depicted in FIGS. 16 and 17, an miR-15a up ligator has been designed to contain two short two base pair regions of self-complementarity. An miR-15a down ligator of an embodiment of the invention is depicted in FIGS. 18 and 19, in which a single three base region of self-complementarity is present.

In certain embodiments, the down ligator includes a modified nucleotide at the 3′ nucleotide to reduce or eliminate the ligation of two down ligators to each other. Suitable modified nucleotides include but are not limited to those that are commercially available: a 3′-amino nucleotide; a dideoxy nucleotide; a 3′-deoxy; a 2′-OH nucleotides; a reverse nucleotide, which could make the 3′ end of the oligo terminate in a 5′-OH; and 3′-alkyl-amino (C3-C10).

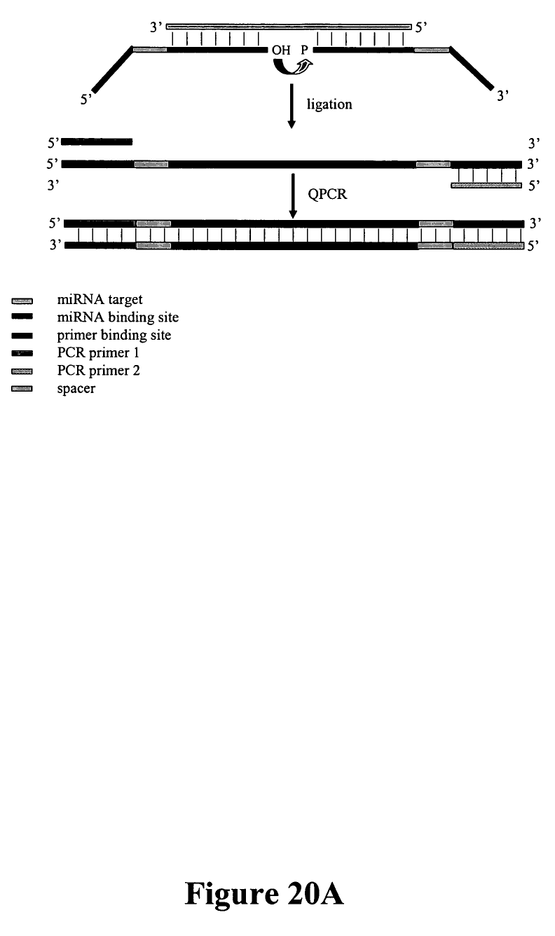

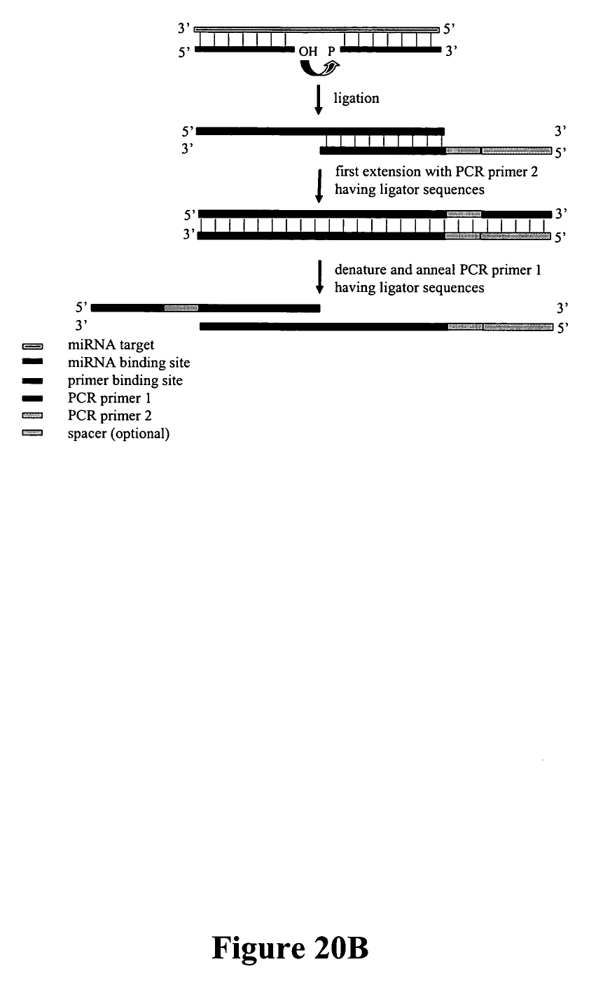

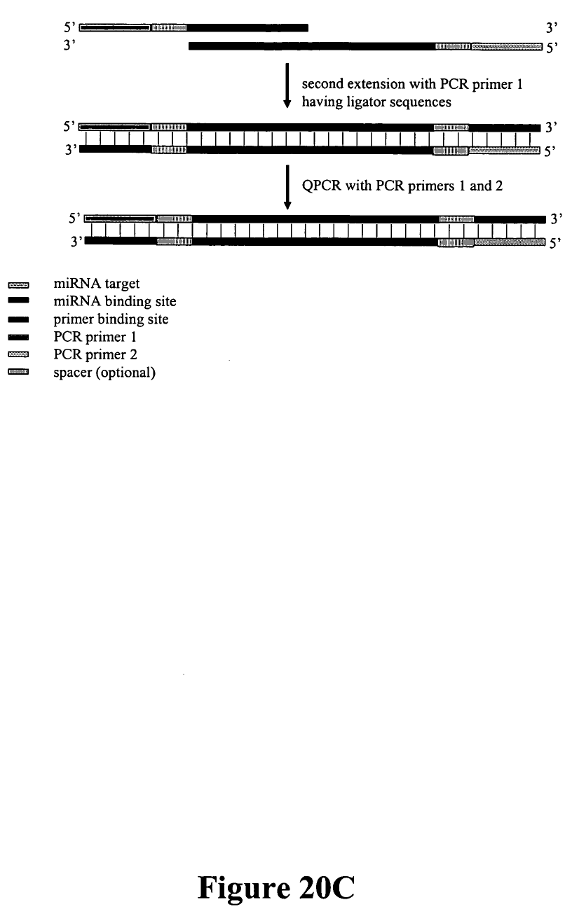

In an alternative embodiment, the up and down ligators comprise or consist of the miRNA binding regions and the up and down ligator sequences having PCR priming sites and optionally spacer sequences are added in a series of extension reactions prior to QPCR (FIGS. 20A-C). This embodiment can be practiced in a series of extension reactions or in a single extension reaction by providing limited amounts of the PCR primers having ligator sequences and non-limited amounts of the PCR primers 1 and 2. Alternatively, the PCR primers having ligator sequences can be used in non-limited amounts to detect the ligation product. A potential advantage of this method is the lack of interaction between the portion of the ligators comprising the PCR priming sites and the spacer with non-target miRNA during the ligation reaction. One having the benefit of this disclosure will realize that additional alternatives including having either the up or down ligator with the miRNA binding region, the spacer region, and the PCR priming site (or complement thereof) and the other ligator having only the miRNA target binding region. Additional combinations of ligators and/or PCR primers having one or more of the regions (miRNA binding region, spacer region, and PCR binding region (or complement thereof)) are also contemplated.

In an alternative embodiment, the up and down ligators include one or more ribonucleotides. These ribonucleotides may be a single ribonucleotide or multiple ribonucleotides, either adjacent to each other or throughout the ligator. In a preferred embodiment, the 5′ terminus of the down ligator is a ribonucleotide.

Ligator oligonucleotides may be produced by any of the numerous suitable techniques known in the art for producing oligonucleotides of 8-500 nucleotides in length. Thus, they may be produced by full chemical or enzymatic synthesis, by chemical synthesis of portions, then ligation of those portions together, by molecular cloning techniques, or by any combination of those techniques and others known in the art. As mentioned above, a ligator oligonucleotide may be a single molecule or it may be a collection of numerous (e.g., millions) copies of a single molecule. Due to the inefficiencies inherent in all chemical synthesis methods, and the inherent error rate in all biological systems, a particular ligator oligonucleotide may contain variations in the sequences in one or more copies. The presence of some amount of variation does not exclude any ligator oligonucleotide from being encompassed by the term. Rather, as long as a sufficient number of molecules within any one substance referred to as a ligator oligonucleotide exist to effect binding to an miRNA target and ligation to a partner ligator oligonucleotide, the substance qualifies as a ligator oligonucleotide according to the invention.

Ligator oligonucleotides can comprise any nucleotide base or analog that is suitable for the intended function of the oligonucleotides. Thus, they can comprise DNA bases, RNA bases, or a mixture of one or more of each. They can comprise polyamide nucleotide bases (PNA; also called peptide nucleic acids). They can comprise locked nucleotide bases (LNA). All bases of a ligator oligonucleotides may be of one type of base or analog. Alternatively, a ligator oligonucleotide may comprise one or more of any combination of two or more of these bases or analogs. Thus, a ligator oligonucleotide may comprise all DNA; all RNA; a mixture of DNA and RNA; a mixture of DNA, RNA, PNA; a mixture of DNA and LNA; etc. Each individual base or analog of the oligonucleotide can be interspersed among bases or analogs of another type, or may be present as part of a continuous sequence of like bases or analogs. Thus, block copolymers of mixtures of base or analog types are contemplated by the invention. For example, a ligator oligonucleotide may comprise 30 RNA bases at its 3′ terminus linked to 20 DNA bases at its 5′ terminus. It likewise may contain 30 RNA bases at the 5′ terminus, 10 PNA bases in the center, and 20 DNA bases at its 3′ terminus. Other combinations will be evident to those of skill in the art from the present disclosure and the general knowledge in the art. The composition of each ligator oligonucleotide to be used in a ligation pair can be selected independently from the other.

The next class of nucleic acids provided by the invention are ligation products produced from ligation of two ligator oligonucleotides. The ligation products may be of any length, but are typically in the range of 50-500 nucleotides in length. Certain non-limiting exemplary lengths are discussed above. In some embodiments, the ligation products are from 70 to 100 nucleotides in length. The ligation product can be detected itself by any number of known techniques, or can serve as a template for amplification, digestion and subcloning, or serve other functions in any other technique in which single-stranded nucleic acids can be used. Thus, in embodiments, the ligation product is a labeled product, containing one or more labels or members of a labeling system at one or more points throughout its sequence. Furthermore, the ligation product may be used for any of a number of other purposes, such as use as a molecular weight or luminescence standard, or a positive control for future practice of the method of the invention to detect the particular target miRNA from which the ligator nucleic acid was produced.

The next class of nucleic acids provided by the invention are amplification primers. Amplification primers are any oligonucleotides that can function to prime polymerization of nucleic acids from template nucleic acids. Those of skill in the art are well aware of techniques and considerations for producing amplification primers, including sets of primers that function reliably and robustly in conjunction with each other to form a double-stranded nucleic acid product of interest from the same template. In accordance with the present invention, the amplification primers are designed in conjunction with the amplification primer binding site of the ligator oligonucleotides, and vice versa. While it is envisioned that there are advantages to designing unique or different amplification primer sequences (and corresponding binding sites on the ligator oligonucleotides), it is also envisioned that the use of standard amplification primer sequences, and thus standard amplification binding sequences on ligator oligonucleotides, can be advantageous in providing a single, standard amplification procedure that can be consistently be reproduced reliably, or at least can reduce the amount of variation, regardless of the identity of the target miRNA. Thus, in embodiments, the amplification primers are selected from among those known in the art as useful for high fidelity amplification of nucleic acids of 50-500 nucleotides in length. In other embodiments, the amplification primers are generated based on selected sequences present on the ligator oligonucleotides or are randomly generated and tested for suitability and specificity.

The amplification primers are designed to bind to the amplification binding site of the ligator oligonucleotides with high specificity. In embodiments where amplification is performed using PCR, the amplification primers can be designed to have melting temperatures that are quite high (e.g., 62° C. or above). The length and nucleotide composition of each particular primer is not limited by any factor except that the primer or primers should be selected in conjunction to produce a primer that will function acceptably to amplify the ligation product for which the primer was designed, if such a ligation product is present in the composition into which the primer is combined. In embodiments where the ligation product contains one or more region of secondary structure as a result of the sequences of the ligator oligonucleotides, it is preferred, but not required, that the amplification primers specifically bind to the ligation product at a temperature above the temperature at which the ligation product's secondary structure melts.

Of course, as is known in the art, amplification primers can include sequences other than those involved in binding to a target sequence. Thus, they may include, at the 5′ end, non-binding nucleotides that can serve any number of functions. Included among the functions are: 1) increase in length of the amplified product as compared to the original template (e.g., to provide nucleotides for restriction endonuclease binding), 2) inclusion of a restriction endonuclease cleavage site, 3) provision of a label or substrate for future labeling, 4) provision of sequence for capture or purification, and 5) any other function contemplated by the practitioner. The various considerations for primer length and binding strength are similar to those discussed above with respect to the portion of the ligator oligonucleotides that bind to the miRNA target, and to those considerations known and widely discussed in the art, and thus need not be repeated here. In summary, amplification primers, while not limited in length, nucleotide content, or sequence, will typically be 18-30 bases long, contain 40-60% G+C content, have a melting temperature (Tm) of about 52° C., show no significant homology to genomic sequences of the organism under study, show no significant secondary structures or structures formed between primers (e.g., using Zucker's Mfold program), not have a 3′ thymidine, and not have multiple G or C at the 3′ end. The main consideration is that the primers function to specifically amplify the ligation product.

The next class of nucleic acids provided by the invention are amplification products. The amplification products are the products produced from amplifying the ligation product. These products can be, but are not necessarily, the same molecules as the ligation products. In embodiments where they differ from the ligation products, they may differ in any of number of ways. For example, they may be longer, and include labels, substrates for labels, restriction endonuclease binding/cleavage sites, multiple primer binding sites, detection sites, and/or hydrolysis probe binding sites. Likewise, amplification products may be shorter than the ligation product. Amplification products that are shorter than their template ligation product may still contain one or more nucleotide sequences that are not present in the ligation product template, including, but not limited to, restriction endonuclease binding/cleavage sites, primer binding sites, labels or label substrates, detection sites, and/or hydrolysis probe binding sites. The amplification products, in addition to being useful for detection, and thus an indication of the presence or absence of a target miRNA in a sample of interest, can be used in a similar fashion to the ligation product, as discussed above. Thus, among other things, they may be used as controls for ligation of ligator oligonucleotides, or as controls for detection of miRNA.

The final class of nucleic acids provided by the invention are miRNA to be detected in the sample. The present invention relies on the known sequence of particular miRNA to be detected to specifically detect that miRNA, to detect miRNA with sequence identity to the known miRNA, or to design ligator oligonucleotides to detect the miRNA and/or miRNA having sequence identity to a known miRNA. miRNA molecules can be provided by the invention to serve as, for example, positive controls for ligation, or any other purpose chosen by the practitioner. Numerous miRNA sequences are publicly available, and one of skill in the art may produce any of these using standard molecular biology techniques. Thus, the miRNA of the invention can be any of those disclosed in Table 3, above. Alternatively, it can be any other miRNA known in the art.

In a third aspect, compositions are provided. Typically, the compositions comprise one or more component that is useful for practicing at least one embodiment of the method of the invention, or is produced through practice of at least one embodiment of the method of the invention. The compositions are not limited in their physical form, but are typically solids or liquids, or combinations of these. Furthermore, the compositions may be present in any suitable environment, including, but not limited to, reaction vessels (e.g., microfuge tubes, PCR tubes, plastic multi-well plates, microarrays), vials, ampules, bottles, bags, and the like. In situations where a composition comprises a single substance according to the invention, the composition will typically comprise some other substance, such as water or an aqueous solution, one or more salts, buffering agents, and/or biological material. Compositions of the invention can comprise one or more of the other components of the invention, in any ratio or form. Likewise, they can comprise some or all of the reagents or molecules necessary for ligation of ligator oligonucleotides, amplification of ligation product, or both. Thus, the compositions may comprise ATP, magnesium or manganese salts, nucleotide triphosphates, and the like. They also may comprise some or all of the components necessary for generation of a signal from a labeled nucleic acid of the invention.

A composition of the invention may comprise one or more ligator oligonucleotides. The ligator oligonucleotide may be any ligator oligonucleotide according to the invention, in any number of copies, any amount, or any concentration. The practitioner can easily determine suitable amounts and concentrations based on the particular use envisioned at the time. Thus, a composition according to the invention may comprise a single ligator oligonucleotide. On the other hand, it may comprise two or more ligator oligonucleotides, each of which having a different sequence, or having a different label or capability for labeling, from all others in the composition. Non-limiting examples of compositions of the invention include compositions comprising one or more ligator oligonucleotides, and a sample containing or suspected of containing an miRNA of interest. Other non-limiting examples include compositions comprising one or more ligator oligonucleotides, a sample containing or suspected of containing an miRNA of interest, and at least one ligase, which is capable under the appropriate conditions of catalyzing the ligation of a ligator oligonucleotide to another ligator oligonucleotide. Yet other non-limiting examples of compositions are those comprising one or more ligator oligonucleotides, a sample containing or suspected of containing an miRNA of interest, at least one ligase, and at least one amplification primer. Yet other non-limiting examples include compositions comprising one or more ligator oligonucleotides, a sample containing or suspected of containing an miRNA of interest, at least one ligase, at least one amplification primer, and at least one polymerase, which is capable under appropriate conditions of catalyzing the polymerization of at least one amplification primer to form a polynucleotide. In certain embodiments, the compositions comprise labels or members of a labeling system. In some embodiments, multiple ligator oligonucleotides are present in a single composition, some of which being specific for one particular miRNA species, others being specific for one or more other miRNA species. In embodiments, the compositions comprise two ligator oligonucleotides.

Alternatively, a composition of the invention may comprise a ligation product of two ligator oligonucleotides. The ligation product may be provided as the major substance in the composition, as when provided in a purified or partially purified form, or may be present as a minority of the substances in the composition. The ligation product may be provided in any number of copies, in any amount, or at any concentration in the composition, advantageous amounts being easily identified by the practitioner for each particular purpose to which the ligation product will be applied. Non-limiting examples of compositions of the invention include compositions comprising a ligation product and one or more ligator oligonucleotides, including those that also comprise at least one ligase. Other non-limiting examples include compositions comprising a ligation product and a sample containing or suspected of containing an miRNA of interest. Still other non-limiting examples of compositions comprise a ligation product and at least one amplification primer. Yet other non-limiting examples of compositions of the invention comprise a ligation product, at least one amplification primer, and at least one polymerase. Yet other non-limiting examples include compositions that comprise a ligation product, at least one polymerase, and an amplification product. In embodiments, the composition comprises agarose, polyacrylamide, or some other polymeric material that is suitable for isolating or purifying, at least to some extent, nucleic acids. In embodiments, the composition comprises nylon, nitrocellulose, or some other solid support to which nucleic acids can bind. In some embodiments, the compositions comprise at least one label or member of a labeling system. Two or more different ligation products may be present in a single composition.

Alternatively, a composition of the invention may comprise one or more amplification primers. The primer may be provided as the major component of the composition, such as in a purified or partially purified state, or may be a minor component. The primer may be any amplification primer according to the invention, in any number of copies, any amount, or any concentration. The practitioner can easily determine suitable amounts and concentrations based on the particular use envisioned at the time. Thus, a composition according to the invention may comprise a single amplification primer. It may also comprise two or more amplification primers, each of which having a different sequence, or having a different label or capability for labeling, from all others in the composition. Non-limiting examples of compositions of the invention that comprise amplification primers include compositions comprising one or more amplification primer and a sample containing or suspected of containing an miRNA of interest. Other non-limiting examples include compositions comprising one or more amplification primer, a sample containing or suspected of containing an miRNA of interest, and at least one ligator oligonucleotide. Still other non-limiting examples include compositions comprising at least one amplification primer, at least one ligator oligonucleotide, a sample containing or suspected of containing a target miRNA, and a ligase, which is capable under the appropriate conditions of catalyzing the ligation of a ligator oligonucleotide to another ligator oligonucleotide. Yet other non-limiting examples of compositions are those comprising the components listed directly above, and further comprising at least one polymerase, which is capable under appropriate conditions of catalyzing the polymerization of at least one amplification primer to form a polynucleotide. In further non-limiting examples, compositions may comprise one or more amplification primer and a ligation product. Additional non-limiting examples include compositions comprising at least one amplification primer and an amplification product. In embodiments, the compositions comprise two or more amplification primers that are designed to function together to produce a double-stranded nucleic acid amplification product. In certain embodiments, the compositions comprise labels or members of a labeling system. In some embodiments, multiple amplification primers are present in a single composition, some of which being specific for one particular ligation product, others being specific for one or more other ligation products.

Alternatively, a composition of the invention may comprise an amplification product. The amplification product may be any nucleic acid that is derived (or has ultimately been produced) from a target miRNA through practice of the method of the invention, where the method includes the optional step of amplification of the ligation product. As with other compositions comprising nucleic acids of the invention, compositions comprising an amplification product may comprise it in any number of copies, amount, or concentration. The amplification product may be provided as the major substance in the composition, as when provided in a purified or partially purified form, or may be present as a minority of the substances in the composition. Non-limiting examples of compositions of the invention include compositions comprising an amplification product and a sample containing a target miRNA. Other non-limiting examples include compositions comprising an amplification product and at least two amplification primers. Other non-limiting examples include those in which the composition comprises an amplification product and at least one polymerase. Yet other non-limiting examples include compositions comprising an amplification product and at least one member of a labeling system. Yet other non-limiting examples include compositions comprising an amplification product and at least one ligase. Other non-limiting examples include compositions comprising an amplification product and a ligation product. Further non-limiting examples include compositions comprising a target miRNA, at least one ligator oligonucleotide, at least one ligase, a ligation product, at least one amplification primer, at least one polymerase, and an amplification product. In embodiments, the composition comprises agarose, polyacrylamide, or some other polymeric material that is suitable for isolating or purifying, at least to some extent, nucleic acids. In embodiments, the composition comprises nylon, nitrocellulose, or some other solid support to which nucleic acids can bind. In some embodiments, the compositions comprise at least one label or member of a labeling system. Two or more different amplification products may be present in a single composition.

Compositions of the invention can comprise one or more nucleic acid polymerase. The polymerase can be any polymerase known to those of skill in the art as being useful for polymerizing a nucleic acid molecule from a primer using a strand of nucleic acid as a template for incorporation of nucleotide bases. Thus, it can be, for example, Taq DNA polymerase, Pfu DNA polymerase, Pfx DNA polymerase, Tli DNA polymerase, Tfl DNA polymerase, klenow, T4 DNA polymerase, T3 RNA polymerase, T7 RNA polymerase, and SP6 RNA polymerase, or combinations thereof.

In a fourth aspect, kits are provided. Kits according to the invention provide at least one component that is useful for practicing at least one embodiment of the method of the invention. Thus, a kit according to the invention can provide some or all of the components necessary to practice at least one embodiment of the method of the invention. In typical embodiments, a kit comprises at least one container that contains a nucleic acid of the invention. In various specific embodiments, the kit comprises all of the nucleic acids needed to perform at least one embodiment of the method of the invention.

Kits are generally defined as packages containing one or more containers containing one or more nucleic acids or compositions of the invention. The kits themselves may be fabricated out of any suitable material, including, but not limited to, cardboard, metal, glass, plastic, or some other polymeric material known to be useful for packaging and storing biological samples, research reagents, or substances. The kits may be designed to hold one or more containers, each of such containers being designed to hold one or more nucleic acids, compositions, or samples of the invention. The containers may be fabricated out of any suitable material including, but not limited to, glass, metal, plastic, or some other suitable polymeric material. Each container may be selected independently for material, shape, and size. Non-limiting examples of containers include tubes (e.g., microfuge tubes), vials, ampules, bottles, jars, bags, and the like. Each container may be sealed with a permanent seal or a recloseable seal, such as a screw cap. One or more of the containers in the kit may be sterilized prior to or after inclusion in the kit.

In certain embodiments, the kit comprises at least two ligator oligonucleotides. These oligonucleotides may be provided separately in different containers or together in a single container. Likewise, multiple containers may be provided, each container one, the other, or both of the ligator oligonucleotides. In embodiments, the kit comprises multiple different ligator oligonucleotides, which can be used to detect the presence of two or more different miRNA targets. In certain configurations of the kit, the ligator oligonucleotides are provided in multiple compositions, each composition comprising two ligator oligonucleotides necessary for detection of a particular target miRNA.

In certain embodiments, the kit comprises at least two ligator oligonucleotides for detection of a particular target miRNA, and further comprises at least one ligase that is capable of ligating the two ligator oligonucleotides together to form a ligation product. In various configurations, the ligator oligonucleotides are provided separately in separate containers or together in a single container. Furthermore, multiple containers containing the various oligonucleotides and ligases can be provided, each independently containing one or more of the oligonucleotides and ligases.

In embodiments, the kit comprises one or more PCR primers. Thus, in embodiments, the kit comprises two PCR primers. In other embodiments, the kit comprises at least two ligator oligonucleotides, at least one ligase, and at least one synthetic miRNA. In yet other embodiments, the kit comprises at least one ligation product, at least one PCR primer (for example, two primers), and at least one polymerase. It yet other embodiments, the kit comprises at least two ligator oligonucleotides, at least one ligase, and at least one DNA ligation template, which comprises the sequence of at least one miRNA.

In certain embodiments, the kit comprises at least two ligator oligonucleotides for detection of a particular target miRNA, at least one ligase that is capable of ligating the two ligator oligonucleotides together to form a ligation product, and at least two amplification primers that can amplify a ligation product. In yet other embodiments, the kit comprises at least two ligator oligonucleotides for detection of a particular target miRNA, and at least two amplification primers that specifically amplify a ligation product produced from ligation of the two ligator oligonucleotides.

In various configurations of the kit, at least one polymerase is included.

In certain configurations of the kit, one or more ligation products specific for pre-defined miRNA are provided. These can be used, for example, as positive control reagents for monitoring of the assay. In configurations of the kit, one or more amplification products may be included.

The kit of the invention may include one or more other components or substances useful in practicing the methods of the invention, such as sterile water or aqueous solutions, buffers for performing the various reactions involved in the methods of the invention, and/or reagents for detection of ligation or amplification products. Thus, in embodiments, the kit comprises one or more polymerase for amplification of a ligation product. In embodiments, it comprises one or more ligases for ligation of ligator oligonucleotides. It also can comprise some or all of the components, reagents, and supplies for performing ligation and amplification according to embodiments of the invention. In embodiments, it includes some or all of the reagents necessary for performing QPCR.

### EXAMPLES

The invention will be further explained by the following Examples, which are intended to be purely exemplary of the invention, and should not be considered as limiting the invention in any way.

### Example 1

### Ligation Reactions Using Synthetic RNA Templates

For gel analysis, ligation reactions were performed in 50 millimolar (mM) Tris-HCl, pH 7.5, 5 mM dithiothreitol (DTT), 15 micromolar (uM) adenosine triphosphate (ATP), 4.5 mM MgCl₂, 25 mM sodium chloride (NaCl), 30 mM potassium chloride (KCl), 0.1 or 0.4 uM each ligator oligonucleotide, 0.1 uM synthetic RNA template, and 10 U T4 DNA ligase (Stratagene). Ligation components (except the T4 DNA ligase) were combined and incubated at 80° C. for 3 min and 16° C. for 5 min. The T4 DNA ligase was added and the ligation reactions were incubated at 23° C. for 2 hours. After 2 hours, the ligation reactions were terminated by heating at 65° C. for 20 minutes and stored at 6° C. until further analysis.

For QPCR analysis, ligation reactions were performed in 50 mM Tris-HCl, pH 7.5, 5 mM dithiothreitol (DTT), 15 uM adenosine triphosphate (ATP), 4.5 mM MgCl₂, 25 mM sodium chloride (NaCl), 30 mM potassium chloride (KCl), either 0.1 or 0.4 uM each ligator oligonucleotide, and either 4 or 10 U T4 DNA ligase (Stratagene). The amount of template was varied in many of the reactions (generally 10² to 10⁸ copies of miRNA template or 75 or 100 ng total RNA) and the reaction may have included Torulla yeast RNA (Ambion). Ligation components (except the T4 DNA ligase) were combined and incubated at 80° C. for 3 min and 16° C. for 5 min. The T4 DNA ligase was added and the ligation reactions were incubated at 23° C. or 30° C. for 2 hours. After 2 hours, the ligation reactions were terminated by heating at 65° C. for 20 minutes and stored at 6° C. until further analysis.

### Example 2

### Ligation Reactions Using miRNA Templates from Cell Samples

For QPCR analysis, ligation reactions were performed as described above, the amount of template was varied in the reactions from 75 to 100 ng. In most experiments, Torulla yeast RNA (Ambion) was added to the reactions to maintain a constant total RNA concentration. It should be noted that the percentage of miRNA in the RNA samples isolated from cells may vary depending upon the method used. Because the samples may comprise more than miRNA, results with these samples might not be accurate indicators of the sensitivity of the ligation-QPCR assay. Ligation components (except the T4 DNA ligase) were combined and incubated at 80° C. for 3 min and 16° C. for 5 min prior to adding the ligase. Ligation reactions were incubated at 23° C. for 2 hours. After 2 hours, the ligation reactions were terminated by heating at 65° C. for 20 minutes and stored at 6° C.

### Example 3

### Analysis of Ligation Reactions

For gel analysis, 10 microliters (ul) of the 20 ul ligation reaction was combined with an equal volume of Novex® TBE-Urea Sample Buffer (2×) (Invitrogen), incubated at 70° C. for 3 min, and stored on ice. The samples were loaded into the wells of a 15% (w/v) TBE-Urea gel and the nucleic acids separated by electrophoresis at 180V until the bromophenol blue dye front was ⅔ to ¾ the length of the gel. The nucleic acids were then stained with SYBR Gold (Molecular Probes) and visualized with the Eagle Eye® II System (Stratagene) according to the manufacturer's recommended conditions.

For QPCR analysis, ligation reactions were diluted 1:10 in water and 2.5 ul of the diluted ligation was added to each QPCR. QPCR was performed using the Brilliant® SYBR® Green QPCR Master Mix (Stratagene) according to the manufacturer's recommended reaction and cycling conditions. The reaction conditions were as follows (25 ul reaction volume): 1× Brilliant® SYBR® Green QPCR Master Mix, 125-150 nanomolar (nM) PCR primer 1, 125-150 nM PCR primer 2, 30 nM ROX (reference dye, Stratagene), and, optionally, 0.5 units (U) uracil-N-glycosylase (UNG; Stratagene), and 1.75 nanograms (ng) Torulla yeast RNA (Ambion). The cycling conditions were: step 1: 1 cycle of 50° C. for 2 minutes (min) (UNG treatment); step 2: 1 cycle of 95° C. for 10 min (hot start), and step 3: 40 cycles of 95° C. for 30 seconds (sec); 55° C. for 60 sec; 72° C. for 30 sec (amplification). A dissociation curve was generated by: step 1: one cycle of 95° C. for 60 sec and ramp down to 55° C. for 30 sec and step 2: ramp up 55° C. to 95° C. (at a rate of 0.2° C./sec). The Mx3000™ m real-time PCR system (Stratagene) was used for thermal cycling and to quantitate the fluorescence intensities during QPCR and while generating the dissociation curve.

If desired, further validation of the miRNA templates amplified can be performed by restriction digestion of the QPCR products at restriction sites prior to visualizing by gel electrophoresis. For example, let-7d digests with Mnl I, miR-16 digests with Ssp I, miR-23b digests with BsaJ I, and miR-125b digests with Spe I. The restriction digestion products are detected by gel electrophoresis as described above. If desired, unique restriction sites can be included when designing the ligators for each miRNA to facilitate confirmation of the QPCR product identity.

### Example 4

### QPCR Testing and Validation

PCR primers for use in the Examples were empirically tested to determine if they would not generate PCR products in the presence of human genomic DNA and in the absence a sequence representing the ligated ligators. No PCR product was detected in selected primers.

QPCR positive control DNA templates were also designed and tested. A single-stranded DNA representing the ligation products of each miRNA tested (Table 3) was used to test various QPCR conditions and to generate standard curves. Two different positive control templates for each miRNA were generated. One positive control (DNA template) consisted of guanidine, adenine, thymidine, and cytosine. The other positive control (DNA template with dUTP) consisted of guanidine, adenine, uracil, and cytosine. When dUTP was used instead of dTTP in the DNA template, incubation with Uracil-N-glycosylase (UNG) prior to QPCR could prevent the subsequent amplification of dU-containing PCR products. UNG acts on single- and double-stranded dU-containing DNA by hydrolysis of uracil-glycosidic bonds at dU-containing DNA sites. When this strategy was used, cross contamination of samples with the dUTP-containing DNA template was eliminated. It should be noted that UTP in the miRNA templates is not hydrolyzed when incubated with UNG.

### Example 5

### miRNA Sources

RNA samples enriched for small RNA, including miRNA, were generated from adenocarcinoma cervical cells (HeLa S3 cells; CCL 2.2; American Type Culture Collection (ATCC)) using the mirVana™ miRNA Isolation Kit (Ambion). The HeLa S3 cells were grown to approximately 80% confluence in Ham's F12K medium with 2 mM L-glutamine adjusted to contain 1.5 g/L sodium bicarbonate, and 10% (v/v) fetal bovine serum (FBS, ATCC) at 37° C. in 5% (v/v) carbon dioxide. RNA, enriched for miRNA, was isolated according to the manufacturer's protocol.

Total RNA samples comprising miRNA were isolated from various cell lines derived from brain, breast, liver, cervix, testis, skin, B lymphocytes, T lymphoblasts, macrophages, and connective tissue using the Absolutely RNA® Miniprep Kit (Stratagene) according to the manufacturer's protocol. The cells were grown to approximately 80% confluence in Dulbecco's minimum essential medium (Invitrogen) containing glucose, penicillin, streptomycin and 10% (v/v) FBS at 37° C. in 5% (v/v) carbon dioxide.

Additionally, miRNA were detected in the Universal Human Reference RNA, a mixture of total RNA isolated from 10 different cell lines (Stratagene; Novoradovskaya, N. M. L. Whitfield, L. S. Basehore, A. Novoradovsky, R. Pesich, J. Usary, M. Karaca, W. K. Wong, O. Aprelikova, M. Fero, C. M. Perou, D. Botstein, and J. Braman. 2004. BMC Genomics. 5:1-16).

Synthetic RNA templates representing miRNA (Tables 3 and 4) were synthesized by various commercial companies including Integrated DNA Technologies (IDT) and Operon using the standard oligonucleotide phosphoramidite synthetic chemistry (McBride, L. J. and M. H. Caruthers. 1983. An investigation of several deoxynucleoside phosphoramidites useful for synthesizing deoxyoligonucleotides. Tetrahedron Lett. 24:245-248) and purified by high performance liquid chromatography (HPLC).

TABLE 4

Nucleotide Sequences of miRNA, up and down ligators,
PCR primers, and DNA templates

| | | SEQ |
|---|---|---|
| | | ID |
| Description | Nucleotide Sequence (5′-3′) | NO: |
| Primer 1 | TAACGCACAGATACGACT | |
| Primer 2 | CATAGCTTGATCGATTATC | |
| let-7d DNA | TAACGCACAGATACGACTAGAGTTCACACTATGCAACCTACTACCT | |
| template | CTTGCTACCTGAGATAATCGATCAAGCTATG | |
| Hsa-let-7d | rArGrArGrGrUrArGrUrArGrGrUrUrGrCrArUrArGrU | |
| miRNA | | |
| let-7d up | TAACGCACAGATACCACTAGAGTTCACACTATGCAACCT | |
| ligator | | |
| let 7D up | TAACGCACAGATACGACTAGAGTGTTGAATAGATCACACTATGCAA | |
| ligator with 8 | CCT | |
| base hairpin | | |
| Let-7d up | TAACGCACAGATACGACTAGAGTGTTGAATAGTTCACACTATGCAA | |
| ligator with 9 | CCT | |
| base hairpin | | |
| let-7d DNA | TAACGCACAGATACGACTAGAGTTCACACTATGCAACCTACTACCT | |
| template with | CTTCAGAGCATTCTACTAAGTCACTGAGATAATCGATCAAGCTATG | |
| hydrolysis | | |
| probe binding | | |
| site | | |
| let-7d down | [Phos]ACTACCTCTTCAGAGCATTCTACTAAGTCACTGAGATAATC | |
| ligator with | GATCAAGCTATG | |
| hydrolysis | | |
| probe binding | | |
| site | | |
| Let-7d | FAM-GTGACTTAGTAGAATGCTCTG-BHQG1 | |
| hydrolysis | | |
| probe with 5′- | | |
| 6FAM and 3′- | | |
| BHQG1 | | |
| miR-15a DNA | TAACGCACAGATACGACTAGAGTTCCACACAAACCATTATGTGCTG | |
| template | CTAAACTACCTGAGATAATCGATCAAGCTATG | |
| Hsa-miR-15a | rUrArGrCrArGrCrArCrArUrArArUrGrGrUrUrUrGrUrG | |
| miRNA | | |
| miR-15a up | TAACGCACAGATACGACTAGAGTTCCACACAAACCATT | |
| ligator | | |
| miR-15a down | [Phos]ATGTGCTGCTAAACTACCTGAGATAATCGATCAAGCTATG | |
| ligator | | |
| miR-16 DNA | TAACGCACAGATACGACTAGAGTTCCACGCCAATATTTACGTGCTG | |
| template | CTAAACTACCTGAGATAATCGATCAAGCTATG | |
| Hsa-miR-16 | rUrArGrCrArGrCrArCrGrUrArArArUrArUrUrGrGrCrG | |
| miRNA | | |
| miR-16 up | TAACGCACAGATACGACTAGAGTTCCACGCCAATATTTA | |
| ligator | | |
| miR-16 down | [Phos]CGTGCTGCTAAACTACCTGAGATAATCGATCAAGCTATG | |
| ligator | | |
| miR-125b DNA | TAACGCACAGATACGACTAGTATTCCTCACAAGTTAGGGTCTCAGGGAAA | |
| template | CTACATCAGATAATCGATCAAGCTATG | |
| Hsa-miR-125b | rUrCrCrCrUrGrArGrArCrCrCrUrArArCrUrUrGrUrGrA | |
| miRNA | | |
| miR-125b up | TAACGCACAGATACGACTAGTATTCCTCACAAGTTAGG | |
| ligator | | |
| miR-125b down | [Phos]GTCTCAGGGAAACTACATCAGATAATCGATCAAGCTATG | |
| ligator | | |
| let-7a miRNA | rUrGrArGrGrUrArGrUrArGrGrUrUrGrUrArUrArGrUrU | |
| let-7b miRNA | rUrGrArGrGrUrArGrUrArGrGrUrUrGrUrGrUrGrGrUrU | |
| let-7c miRNA | rUrGrArGrGrUrArGrUrArGrGrUrUrGrUrArUrGrGrUrU | |
| let-7e miRNA | rUrGrArGrGrUrArGrGrArGrGrUrUrGrUrArUrArGrU | |

[Phos]= phosphate
rG = guanidine
rA = adenosine
rC = cytosine
rT = thymidine
G = deoxyguanidine
A = deoxyadenine
T = deoxythymidine
C = deoxycytosine
U = uracil
FAM = fluorescein (Biosearch Technologies)
BHQ1 = Black Hole Quencher ™-1 dye (Biosearch Technologies)

### Example 6

### Generation of Standard Curves

A standard curve is useful in optimizing QPCR conditions, testing the effect of ligation reaction components on QPCR efficiency, determining the lower and upper detection limits, determining the QPCR efficiencies over different ranges of template input, and for comparison in determining the concentrations of miRNA in test samples. Thus, standard curves were generated for analysis of amplification of exemplary miRNA according to methods of the invention.

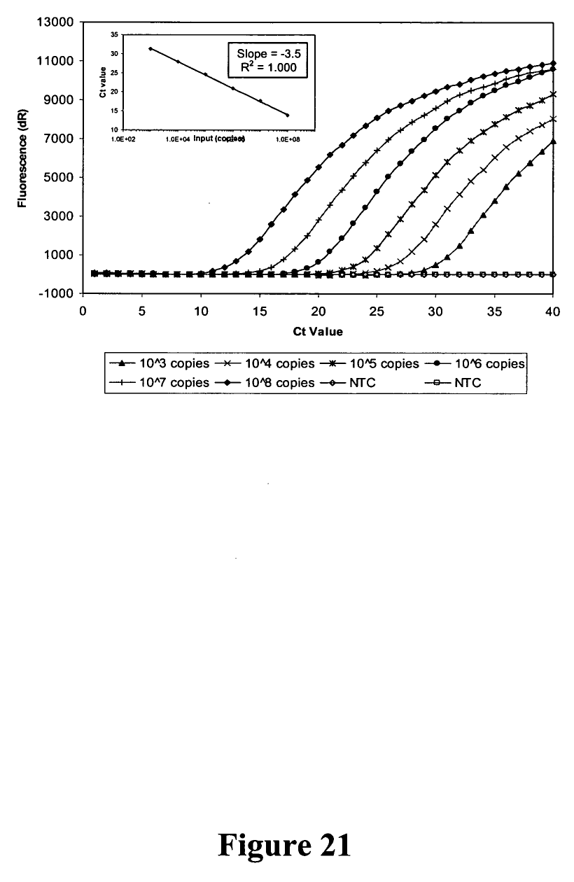

For example, a standard curve was generated using 10³ to 10⁸ molecules of the let-7d DNA template with dUTP in QPCR (FIG. 21). As can be seen from the Figure, the standard curve is linear over 5 logs with a Pearson's correlation coefficient (R²) of 1.000 and a slope of −3.5. The linearity of the standard curve and the high correlation coefficient indicate highly similar QPCR efficiencies over a wide range of input DNA template. Similar standard curves were generated with each DNA template corresponding to a different miRNA indicating similar amplification efficiencies of the template representing the ligated ligators. When standard curves generated with the DNA template with dUTP were used to estimate the miRNA copy number, the estimated copy number from template-independent ligation (represented by the ligators plus T4 DNA ligase in the absence of template) was subtracted from the estimated copy number from template-dependent ligations (represented by the ligators plus T4 DNA ligase in the presence of template).

A standard curve is also useful in optimizing ligation conditions by testing the effect of the reaction components and conditions on ligation efficiency, in determining the lower and upper detection limits of the assay, and for comparison in determining the miRNA copy number in test samples. Accordingly, standard curves were generated to analyze ligation reactions according to methods of the invention.

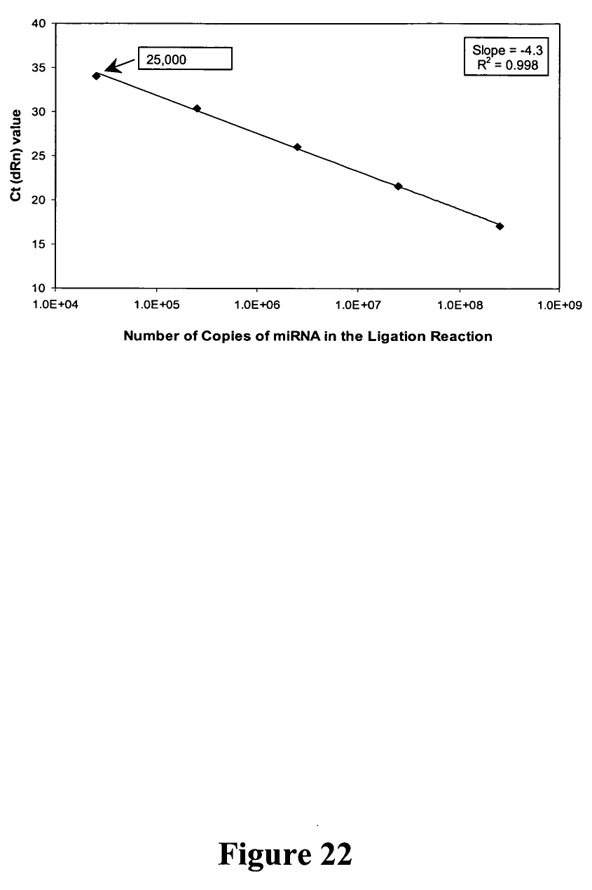

In this example, a standard curve was generated using 2.5×10⁴ to 2.5×10⁸ molecules of the let-7d miRNA template in the ligation-QPCR assay (FIG. 22). The standard curve is linear over 4 logs with a Pearson's correlation coefficient (R²) of 0.998 and a slope of −4.3. The linearity of the standard curve and the high correlation coefficient indicate similar ligation and QPCR efficiencies over a wide range of input miRNA template. The result indicates a lower detection limit of 2.5×10⁴ let-7d miRNA molecules.

When the standard curve is generated with the let-7d miRNA template, subtraction of the background resulting from template-independent ligation (represented by the ligators plus T4 DNA ligase in the absence of template) is not required prior to its use to determine the miRNA copy number in test samples. However, the background should be set as the lower limit of sensitivity of the assay and hence any values that fall below the background should not be considered accurate.

### Example 7

### Detection of let-7d and miR-16 in HeLa miRNA Sample Using the Ligation-QPCR Assay of the Invention

The ligation-QPCR assay of one embodiment of the invention was used to detect let-7d and miR-16 in a sample that had been enriched for low molecular weight RNA, including miRNA, from HeLa S3 tissue culture cells. The method used to generate this sample uses differential binding of RNA to a matrix to separate long and short RNA. The resultant sample was not, however, analyzed to determine the effectiveness of the separation of the long and short RNA.

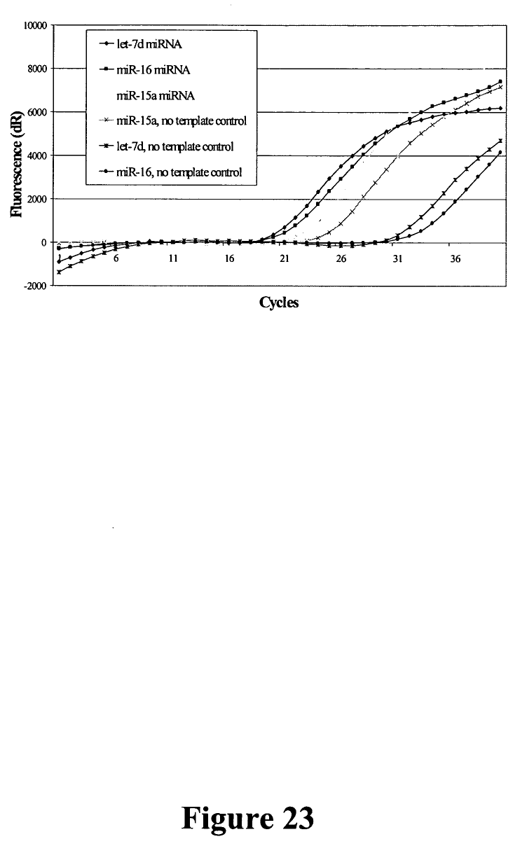

Let-7d and miR-16 were detected in 75 ng sample that was enriched for miRNA sequences from HeLa cells (HeLa miRNA) by the ligation-QPCR method of this invention (FIG. 23). The Ct values were compared to a standard curve generated with the let-7d mRNA template as described above in to estimate the number of let-7d, miR-16, and miR-15a (Table 5).

### Example 8

### Relative Amounts of let-7d, miR-16, and miR-15a miRNA Detected in Ligation-QPCR and microRNA Microarray Assays

A microRNA microarray was also used to quantitate the presence of let-7d, miR-16, and miR-15a in the HeLa miRNA sample. The microRNA microarray and labeled miRNA were prepared and processed as previously described (Thomson, M. J., J. Parker, C. M. Perou, and S. M. Hammond. 2004. A custom microarray platform for analysis of microRNA gene expression. Nature Methods. 1:47-53).

Numerous miRNA, including let-7d, miR-16, and miR-15a, were detected in 750 ng of the HeLa miRNA. The amount of hybridization of the fluorescence-labeled miRNA was quantitated by scanning the array using the GenePix® 4000A scanner and analyzed using GenePix Pro 3.0 (Axon Instruments). The fluorescence values are the average of the median less the local background for two duplicate spots with the corresponding standard deviations (Table 5).

TABLE 5
Relative Abundances of let-7d, miR-16, and miR-15a miRNA
Detected in Ligation-QPCR and microRNA Microarray Assays

| | | Fluorescence | | Relative |
|---|---|---|---|---|
| | Estimated copy | intensity by | Relative | abundance |
| | number by | microRNA | abundance to | to miR- |
| | ligation-QPCR | microarray | mi-15a by | 15a by |
| miRNA | assay | assay | ligation-QPCR | microarray |
| let-7d | 1.65 × 10⁷ | 21,545 +/− | 6.5 | 25.2 |
| | | 6,706 | | |
| miR-16 | 9.70 × 10⁶ | 2,661 +/− 322 | 3.8 | 3.2 |
| miR-15a | 2.53 × 10⁶ | 845 +/− 119 | 1.0 | 1.0 |

Estimates of the copy number of let-7d, miR-16, and miR-15a were made by using a standard curve as described above. However, microarray assays do not allow for inclusion of a standard curve, therefore, an estimate of the copy number of let-7d, miR-16, and miR-15a cannot be made from microarray results. Therefore, the relative amounts of let-7d, miR-16, and miR-15a detected by the ligation-QPCR and microRNA microarray assays were compared (Table 5).

The similar ratios for miR-16 as determined by the ligation-QPCR and microRNA microarray methods is further validation of the ligation-QPCR method. The underestimation of let-7d by the ligation-QPCR method when compared to the microarray method may indicate that either or both methods are not distinguishing between the different members of the let-7 family and thus are not absolutely specific to let-7d.

### Example 10

### Detection of let-7d, miR-15a, and miR-16 in Various Total RNA Samples

To demonstrate that the method of this invention can be applied to more than just samples enriched for miRNA, the amount of let-7d, miR-15a, and miR-16 miRNA was detected in various samples comprising total RNA. As previously discussed, the Absolutely RNA® Miniprep Kit was not designed to isolate RNA of <100 nucleotides, however, we have detected miRNA in total RNA isolated using this kit. This is likely due to the interaction between a miRNA and its target mRNA resulting in their co-isolation. It is therefore also likely that more miRNA was originally present in the cells and was not isolated. While the use of this kit may result in a low efficiency in the isolation of miRNA, it was still surprisingly satisfactory for our purposes. The kit uses DNase to hydrolyze genomic DNA and thereby ensure its absence in the total RNA. Since the genomic DNA includes the sequences transcribed into miRNA, its presence may lead to incorrect results.

let-7d, miR-15a, and miR-16 were detected in 100 ng total RNA isolated from various cell lines and in UHRR by the methods of this invention. In this example, ligators were annealed to the miRNA present in 100 ng total RNA and ligated as described above. Ligation of the ligators was then detected by QPCR as described above.

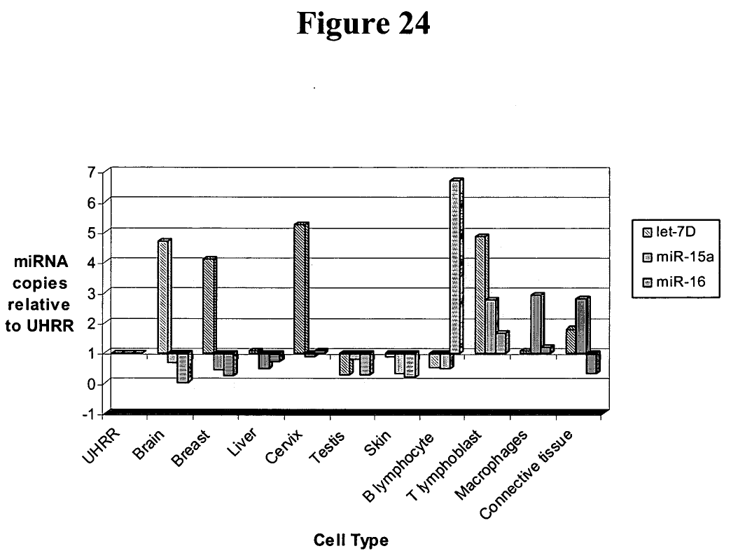

The resultant Ct values of each cell line and the blend of 10 cell lines, UHRR, were compared to a standard curve and the copy number of each miRNA was estimated (FIG. 24). The value for template-independent ligation represented by the samples without template but with each of the ligators and ligase was subtracted from each value. The calculated values revealed a broad range of values that were unique for each miRNA. The broad range of values also indicated that the method of this invention is capable of detecting miRNA over a broad range of input molecules. Additionally, the results indicated that miRNA could be detected in samples other than those enriched for miRNA.

### Example 11

### Detection of let-7d Using Ligators with Probe Binding Sites and Hairpin

Ligator designs are contemplated which may increase annealing specificity and/or ligation efficiency. When the ligators are incubated with RNA from cells, they may anneal to target or non-target RNA (or DNA, if present) anywhere along the ligator. Since the miRNA may be a small percentage of the RNA present in the cell sample, a method which increases the likelihood that the ligators specifically anneal to the miRNA is desirable.

One such method is to use sequences which introduce self-complementarity at the 3′ and/or 5′ ends of the up and down ligators, respectively. The presence of the self-complementarity enables the 3′ or 5′ ends of the up and down ligators, respectively, to fold back on themselves and form a hairpin loop comprising a stem and a loop. The loop does not include self-complementarity and therefore is not designed to anneal to any other nucleotides in the ligator. The stem includes self-complementarity and therefore is designed to anneal to other nucleotides in the ligator. The Tm of the hairpin loop is controlled by varying the number of bases having self-complementarity, varying the number of bases that anneal within the stem structure, varying the positions of bases that anneal within the stem structure, and varying the identities of the bases that anneal. For example, a G annealing to a C will have a Tm of about 4° C. while an A annealing to a T will have a Tm of about 2° C. More precise estimations of the Tm can be obtained using Mfold (Zucker, above), but are not necessary.

The hairpin ligator will exist in two different conformations, one conformation is with regions of self-complementarity annealed to form a hairpin loop and the other conformation is with the regions of self-complementarity not annealed. When the regions of self-complementarity are annealed, the ligator is less likely to anneal to the target miRNA. When the regions of self-complementarity are not annealed, the ligator is more likely to anneal to the target miRNA.

The conformation of the ligator is controlled by the design methods described above and by the ligation reaction conditions. Under reactions conditions below the Tm of the regions of self-complementarity, the ligator will exist primarily in the hairpin conformation. Under reaction conditions above the Tm of the regions of self-complementarity, the ligator will not exist primarily in the hairpin conformation. Under reaction conditions at or near the Tm, the ligator will exist in both the hairpin and non-hairpin conformations.

The self-complementarity hairpin regions were designed to have a lower Tm than the Tm of the portion that is complementary to the miRNA annealed to its target miRNA (see, For example, FIGS. 4-19). The Tm of the self-complementary hairpin region can be estimated using Mfold (Zucker, above). The settings used in Mfold can be: a folding temperature of 23° C., a Na⁺ concentration of 55 mM, and a Mg⁺⁺ concentration of 4.5 mM. The results of the Mfold program are in ΔG. ΔG is the minimum free energy. RNA for which the native state (minimum free energy secondary structure) is functionally important (for example: tRNA, small nucleolar spliceosomal RNA, 5S rRNA) will have lower folding energy than random RNA of the same length and dinucleotide frequency. Thus, the lower the ΔG, the more stable the structure. The Tm of the portion that is complementary and annealed to its target miRNA can be estimated using MELTING (Le Novere, N., above). The settings used in the MELTING program were: nearest neighbor predictions as defined in Sugimoto N, Nakano S, Katoh M, Matsumura A, Nakamuta H, Ohmichi T, Yoneyama M, Sasaki M. 1995. Thermodynamic parameters to predict stability of RNA/DNA hybrid duplexes. Biochemistry. 34(35):11,211-11,216 and the default salt correction. The results of the MELTING program are in ° C. See Table 2, above, for example.

The let-7d up ligators with either the 8 or 9 base hairpin have a lower ΔG than the let-7d up ligator without a hairpin indicating the higher stability of the folded than the linear form of the ligator.

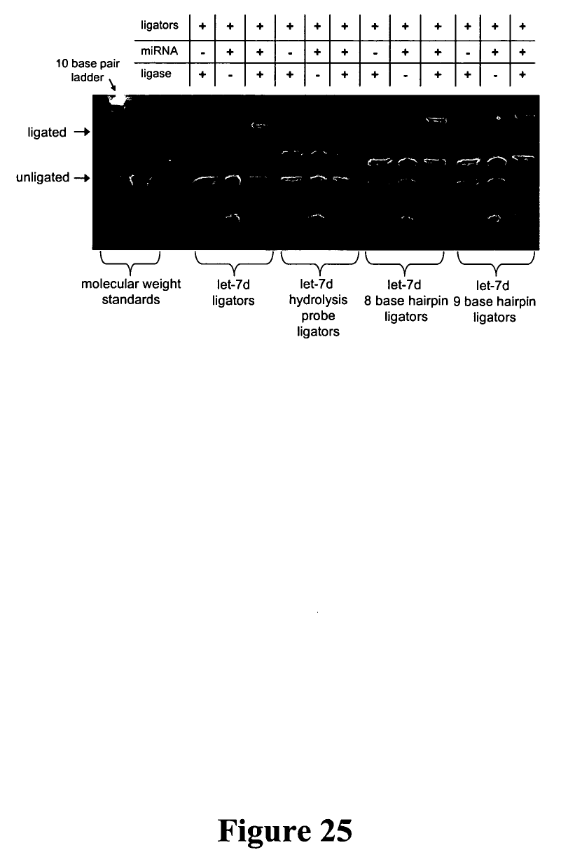

The synthetic let-7d miRNA was detected using hairpin ligators with either 8 or 9 bases of self-complementarity and detected by gel electrophoresis by the methods of this invention (FIG. 25). The ligation products are clearly evident in those samples containing the let-7d miRNA template, ligators, and ligase indicating that these ligators anneal and are ligated in the presence of the let-7d miRNA template. In addition, ligators having either 8 or 9 bases in the hairpin generate similar amounts of ligation product and are therefore ligated with similar efficiencies.

### Example 11

### Determining the Effect of Additives on QPCR

Perfect Match® PCR Enhancer (Stratagene) has been shown to increase yield and specificity of primary PCR amplification products, minimize the formation of poorly matched primer-template complexes, and destabilize many mismatched primer-template complexes. The primary component of Perfect Match® interacts with both DNA and RNA. The use of Perfect Match® may therefore increase specificity in the ligation-QPCR assay.

In order to test any additive for use in this invention, the additive is preferably tested in both the ligation and QPCR. Any additive that has a beneficial effect to the ligation reaction but has an adverse effect on QPCR can be removed from the ligation reaction by purification prior to its addition to the QPCR.

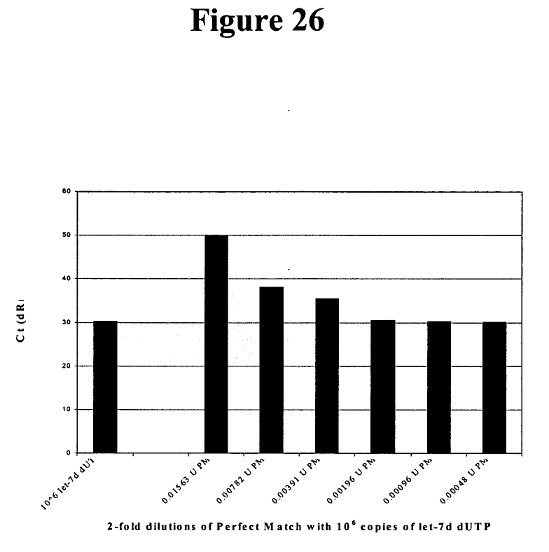

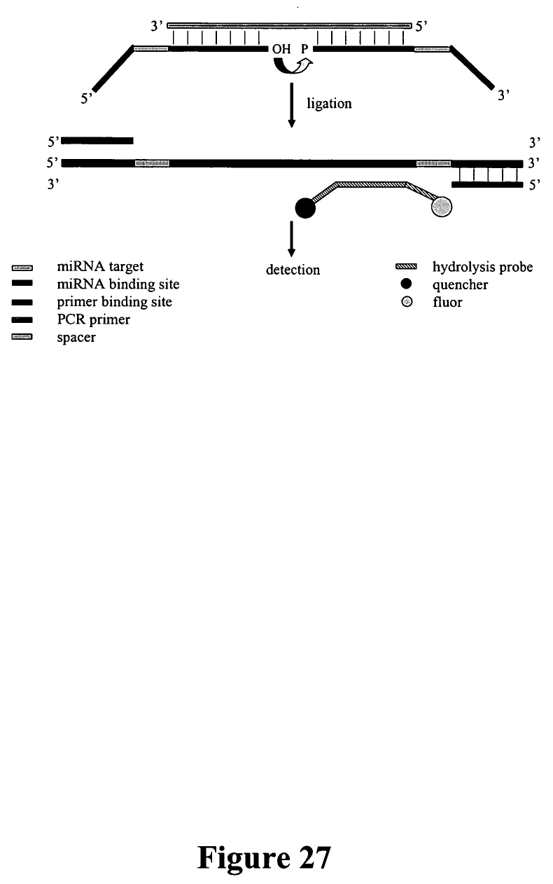

In this example, varying amounts of Perfect Match® were added to QPCR using 10⁶ copies of the let-7d DNA template with dUTP as described above and in the product literature for Perfect Match. The Perfect Match (1 U/ul) was diluted 2-fold in water and 1 ul was added to a 25 ul reaction. The amount of Perfect Match® varied from 1 to 0.00048 U/reaction. The Ct was plotted vs the amount of Perfect Match® (FIG. 26). No Ct was given for QPCR with 1 to 0.03126 U per reaction. As shown in Figure, 10⁶ molecules of the let-7d DNA template with dUTP had a Ct of 30, therefore, samples with higher Cts were inhibited. Samples with a Ct of 30 were not inhibited. As can be seen in FIG. 26, a decrease in Ct from 50 to 30 occurs between samples with 0.01563 and 0.00196 U Perfect Match® per reaction. Samples with less than 0.00196 U Perfect Match® have Cts of 30 and therefore were not inhibited. The addition of Perfect Match® did not appear to enhance the QPCR results in this example as no Ct lower than 30 were observed.

These results are used as a guideline in using Perfect Match® in the ligation reactions to ensure that the amount of Perfect Match® in the ligation reaction added to the QPCR does not inhibit the QPCR. If the amount of Perfect Match® that improves the results of the ligation reaction are not compatible with the QPCR reagents, the ligation reactions can be purified prior to addition to the QPCR to remove the inhibitory effect.

While this experiment yields guidelines on the amount of Perfect Match® to use with the Brilliant® SYBR® Green QPCR Master Mix (Stratagene), one of skill in the art would realize that other QPCR or PCR reagents may yield different results, and that these experiments should be performed with those reagents. Such experiments to optimize other commercially available systems is well within the level of skill of those of skill in the art, and do not require undue experimentation.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A method of detecting the presence or absence of an miRNA in a sample, said method comprising
providing a sample containing or suspected of containing the miRNA,
providing at least two ligator oligonucleotides,
providing at least one ligase,
combining the sample, ligator oligonucleotides, and ligase to form a composition suitable for ligation of the ligator oligonucleotides, and
detecting ligation of the oligonucleotides.

2. The method of claim 1, wherein detecting is by gel electrophoresis and staining of the ligation product.

3. The method of claim 1, further comprising combining at least one amplification primer to a composition comprising the miRNA.

4. The method of claim 3, further comprising exposing a composition comprising the miRNA and the at least one primer to at least one polymerase.

5. The method of claim 1, further comprising amplifying a ligation product produced from the combination of miRNA, ligator oligonucleotides, and ligase.

6. The method of claim 5, wherein amplifying is performed using PCR.

7. The method of claim 6, wherein the PCR is QPCR.

8. A composition comprising at least two ligator oligonucleotides, wherein a first ligator oligonucleotide has a 3′ terminal sequence that can hybridize under stringent conditions to a 5′ terminal sequence of a target miRNA, and where a second ligator oligonucleotide has a 5′ terminal sequence that can hybridize under the same stringent conditions to a 3′ terminal sequence of the target miRNA, such that hybridization of the first and second ligator oligonucleotides to the target miRNA causes the 5′ terminal nucleotide of one ligator oligonucleotide to be adjacent to the 3′ terminal nucleotide of the other ligator oligonucleotide.

9. The composition of claim 8, wherein one or more of the ligator oligonucleotides comprises a sequence that can form a secondary structure.

10. The composition of claim 8, further comprising at least one ligase.

11. The composition of claim 8, further comprising at least one amplification primer.

12. The composition of claim 8, further comprising a sample containing or suspected of containing an miRNA of interest.

13. The composition of claim 8, comprising a sample containing or suspected of containing an miRNA of interest, at least two ligator oligonucleotides, and at least one ligase.

14. The composition of claim 13, further comprising at least one amplification primer and at least one polymerase.

15. The composition of claim 8, further comprising at least one blocking oligonucleotide.

16. A kit comprising, in packaged combination, at least two ligator oligonucleotides.

17. The kit of claim 16, further comprising at least one amplification primer.

18. The kit of claim 16, further comprising at least one ligase.

19. The kit of claim 16, further comprising at least one polymerase.

20. The kit of claim 16, further comprising an miRNA of known sequence.

21. The kit of claim 16, further comprising some or all of the components necessary to perform QPCR.
